(19) United States
(12) Patent Application Publication
(10) Pub. No.: US 2014/0088700 A1
(43) Pub. Date: Mar. 27, 2014

(54) METHOD FOR USING A SILK DERIVED BIORESORBABLE SCAFFOLD IN BREAST RECONSTRUCTION

(71) Applicant: Allergan, Inc., Irvine, CA (US)

(72) Inventors: Enrico Mortarino, Hickory, NC (US); Gregory H. Altman, Arlington, MA (US); Jeff Ippolito, Medford, MA (US)

(73) Assignee: Allergan, Inc., Irine, CA (US)

(21) Appl. No.: 14/028,389

(22) Filed: Sep. 16, 2013

Related U.S. Application Data
(63) Continuation-in-part of application No. 13/306,325, filed on Nov. 29, 2011.
Application No. 13/306,325 is a continuation-in-part of application No. 12/680,404, filed on Sep. 19, 2011.
Application No. 12/680,404 is a continuation-in-part of application No. 13/186,151, filed on Jul. 19, 2011.
Application No. 13/186,151 is a continuation-in-part of application No. 13/156,283, filed on Jun. 8, 2011.

(51) Int. Cl. A61F 2/12

(52) U.S. Cl. CPC A61F 2/12
USPC 623/8

(57) ABSTRACT

A method of using a silk derived medical device in a two stage breast reconstruction procedure.

### CROSS-REFERENCE TO RELATED APPLICATIONS

This application is a continuation-in-part of U.S. patent application Ser. No. 13/306,325, filed Nov. 29, 2011, which is a continuation-in-part of U.S. patent application Ser. No. 13/186,151, filed Jul. 19, 2011, which is a continuation-in-part application of U.S. patent application Ser. No. 13/156,283, filed Jun. 8, 2011, which is a continuation-in-part application of U.S. patent application Ser. No. 12/680,404, filed Mar. 26, 2010, which is itself the United States national stage entry application of the PCT Patent Application No. PCT/US09/63717, filed Nov. 9, 2009, which claims priority to and the benefit of U.S. Provisional Patent Application No. 61/122,520, filed Dec. 15, 2008, all of which applications are expressly incorporated by reference herein in their entireties.

### BACKGROUND

The present invention generally relates to a prosthetic device for tissue repair, and, more particularly, to a surgical silk mesh or scaffold device employing a stable knit structure and a method of using the same in breast cosmetic and surgical procedures, such as in breast augmentation and/or breast reconstruction procedures.

Surgical mesh initially used for hernia and abdominal wall defects are now being used for other types of tissue repair, such as rotator cuff repair, pelvic floor dysfunction, and reconstructive or cosmetic surgeries. It is projected that in 2010, there will be more than 8 million hernia procedures, 800,000 rotator cuff repairs, 3 million pelvic prolapse repairs, 600,000 urinary incontinence repairs, and 1.5 million reconstructive or aesthetic plastic surgeries. Most of these procedures will likely employ implantable surgical mesh devices currently on the market, including: Bard Mesh (polypropylene) by C. R. Bard; Dexon (polyglycolic acid) by Synecture/US Surgical; Gore-Tex (polytetraflouroethylene) by W. L. Gore; Prolene (polypropylene), Prolene Soft (polypropylene), Mersilene Mesh (polyester), Gynemesh (polypropylene), Vicryl Knitted Mesh (polyglactin 910), TVT (polypropylene) by Ethicon; Sparc tape (polypropylene) by American Medical Systems; and IVS tape (polypropylene) by TYCO Healthcare International.

Surgical mesh devices are typically biocompatible and may be formed from bioresorbable materials and/or non-bioresorbable materials. For example, polypropylene, polyester, and polytetraflouroethylene (PTFE) are biocompatible and non-bioresorbable, while polyglactin 910 and polyglycolic acid are biocompatible and bioresorbable.

Though current surgical mesh devices may be formed from different materials, they have various similar physical and mechanical characteristics beneficial for tissue repair. However, despite the benefits provided by current surgical mesh devices, their use may be accompanied by a variety of complications. Such complications, for example, may include scar encapsulation and tissue erosion, persistent infection, pain, and difficulties associated with revision surgery. In addition, the use of an absorbable material may result in reoccurrence due to rapid resorption of the implant material and loss of strength.

Although polypropylene monofilament may be a highly regarded material for surgical mesh devices, polypropylene mesh devices can induce intense scar formations and create a chronic foreign body reaction with the formation of a fibrous capsule, even years after implantation. Minor complaints of seromas, discomfort, and decreased wall mobility are frequent and observed in about half of the patients implanted with polypropylene mesh devices. Moreover, polypropylene generally cannot be placed next to the bowel due to the propensity of adhesion formation.

Although the use of multifilament polyester may improve conformity with the abdominal wall, it is also associated with a variety of disadvantages. For example, higher incidences of infection, enterocutaneous fistula formation, and small bowel obstruction have been reported with the use of multifilament polyester compared to other materials. Indeed, the small interstices of the multifilament yarn make it more susceptible to the occurrence of infection, and thus multifilament polyester is not commonly used within the United States.

The use of polytetraflouroethylene (PTFE) may be advantageous in minimizing adhesions to the bowel. However, the host tissue encapsulates the PTFE mesh, resulting in weak in-growth in the abdominal wall and weaker hernia repair. This material, though not a good mesh material on its own, has found its place as an adhesion barrier.

Absorbable materials, such as Vicryl and Dexon, used for hernia repair have the advantage of being placed in direct contact with the bowel without adhesion or fistula formation. A study has observed that Vicryl has comparable burst strength to nonabsorbable mesh at three weeks but is significantly weaker at twelve weeks due to a quick absorption rate. Meanwhile, the same study observed that Dexon has more in-growth at twelve weeks with less absorption of the mesh. The concern with absorbable meshes is that the rate of absorption is variable, possibly leading to hernia recurrence if the proper amount of new tissue is not there to withstand the physiologic stresses placed on the hernia defect.

A significant characteristic of a biomaterial is its porosity, because porosity is the main determinant for tissue reaction. Pore sizes of >500-600 μm permit in-growth of soft tissue; pore sizes of >200-300 μm favor neo-vascularisation and allow mono-morphological restitution of bony defects; pore sizes of <200 μm are considered to be almost watertight, hindering liquid circulation at physiological pressures; and pores of <100 μm only lead to in-growth of single cell types instead of building new tissues. Finally, a pore size of <10 μm hinders any in-growth and increases the chance of infection, sinus tract formation, and encapsulation of the mesh. Bacteria averaging 1 μm in size can hide in the small interstices of the mesh and proliferate while protected from neutrophilic granulocytes averaging 10-15 μm.

Other important physical characteristics for surgical mesh devices include thickness, burst strength, and material stiffness. The thickness of surgical mesh devices vary according to the particular repair procedure. For example, current surgical mesh device hernia, pelvic floor dysfunction, and reconstructive/cosmetic procedures range in thickness from approximately 0.635 mm to 1.1 mm. For rotator cuff repair, a thickness of 0.4 mm to 5 mm is typically employed.

Intra-abdominal pressures of 10-16 N, with a mean distension of 11-32% results in the need for a surgical mesh with a burst strength that can resist the stress of the inner abdomen before healthy tissue comes into being.

Material stiffness is an important mechanical characteristic for surgical mesh, especially when used for pelvic floor dysfunction, because material stiffness has been associated with the likelihood of tissue erosion. Surgical mesh devices formed from TVT, IVS, Mersilene, Prolene, Gynemesh, Sparc tape, for example, currently have an ultimate tensile strength (UTS) that exceeds the forces exerted by intra-abdominal pressures of 10-16 N. With the low force in the abdomen, the initial stiffness of the material is an important consideration. Moreover, the stiffness may exhibit non-linear behavior most likely due to changes in the fabric structure, e.g., unraveling of the knit, weave, etc. A surgical mesh device of lesser stiffness may help reduce tissue erosion and may conform to the contours of the body more effectively.

There are also considerations associated with surgical procedures for breast reconstruction. Following mastectomy, a woman may choose to have reconstruction using her own tissue (autologous reconstruction) or breast implants. Autologous tissue may closely resemble the look and feel of the native breast; however, some women may want to avoid the scar and donor site morbidity associated with this procedure, and others simply do not have enough tissue to perform autologous reconstruction. Therefore, an increasing number of women are opting for implant reconstruction.

In Europe, one-stage immediate breast implant, or Direct-to-Implant (DTI), reconstructions have been performed and are considered beneficial in that this approach can reduce the number of hospitalizations and the surgical costs associated with multiple stage reconstructions. A study by Plant et al. suggested that single-stage reconstructions can be as effective as two-stages reconstructions. These single-stage implantations are typically subsequent to skin-sparing and nipple-sparing mastectomies and often use the patient's autologous tissue to secure and support the breast implant. Single-stage surgeries using autologous tissue generally see few implant related complications, low capsular contracture gradings and good cosmetic results. Two-stage reconstructions are also performed.

In implant reconstruction, usually a tissue expander (TE) is placed after mastectomy and expanded until the desired pocket size has been attained. The tissue expander is then removed, and a permanent implant is inserted. A recent advancement to this procedure is the addition of material to support implant reconstruction by providing a framework to control the space or position of the tissue expander. A “scaffold” is sutured to the chest wall and anterior rectus abdominus fascia, creating a pocket or hammock for subsequent tissue expander or implant placement. The superior portion of the material is sutured to the inferior aspect of the pectoralis muscle in order to cover the tissue expander. This graft can serve as a protective barrier between the implant and the skin, it can control the position of the implant, and it can decrease the force transmission to the implant itself. Advantages of using this technique include effectively “lengthening” the pectoralis muscle coverage of the tissue expander without compromising lower pole expansion, precise control of the inframammary fold and lateral breast border, and allowance of greater initial fill-volumes. Additionally, grafts can allow the skin envelope to be used before it becomes contracted, which may yield a more natural aesthetic outcome.

AlloDerm, an allogenic acellular dermal matrix (ADM) has been frequently used as the scaffold during breast reconstruction. When used to create complete coverage of the tissue expander, AlloDerm may allow higher initial fill volumes of tissue expanders, more rapid expansion, improved definition of inframammary folds, and may result in less postoperative pain, though Preminger et al. did not find differences in initial fill volume or rate of expansion between an AlloDerm breast reconstruction group and a control group.

The outcomes and complication rates of using acellular cadaveric dermis in staged reconstruction was retrospectively examined by Bindingnavele et al. Complication rates in 65 breasts were 4.6% for seroma, 3.1% for infection, 1.5% for expander removal, and 1.5% for hematoma. Four of 5 patients underwent unplanned radiation therapy after reconstruction with the cadaveric dermis. One of the 5 developed a wound infection that required explantation of the tissue expander.

Complication rates were higher with AlloDerm compared to a control group in some studies, but not in others. Chun et al. found complication rates of 14% for seroma, 9% for infection, and 23% for necrosis but in a comparative study of AlloDerm with a similar allogenic acellular dermal matrix (DermaMatrix), the overall complication rate was 4% in the 50 breasts (25 per each group) with one seroma and one infection cellulitis. The only significant difference found between the two matrices was in the mean number of days in which patients had drains in place (11 for AlloDerm and 13 for DermaMatrix; p=0.02). Another study found 5% postoperative infection with the use of AlloDerm and 5.85% without AlloDerm. In a study of 96 women undergoing two-stage reconstruction using ADM, 11 seromas (7.2%) were identified, with 9 of those undergoing aspiration.

Fifty-eight breasts that underwent reconstruction with crescentric tissue expansion were evaluated by Buck et al. Overall, there were 5 complications (8.6%); 2 tissue expanders became infected, 1 patient developed flap necrosis, 1 patient developed a hematoma, and 1 developed a hematoma. Patients were satisfied with their outcomes.

The logistics of using AlloDerm can be an issue (e.g., it has a shelf-life of only 2 years and requires at least 30 minutes of rehydration before application). In addition, it has been recommended to undergo two saline baths, and costs of ADM may be significant. Other ADMs have appeared on the market. In a small study, NeoForm was evaluated for safety and effectiveness. No complications related to NeoForm were found in 22 patients, and the tissue expansion procedures went as planned. Although some improvement in the logistics may be found with NeoForm (i.e., no need for refrigeration, 5 year shelf-life, and 3 to 5 minutes for rehydration), average follow-up of these patients was relatively short (10.2 months).

### SUMMARY

In view of the disadvantages of current surgical mesh devices, particularly in breast reconstruction procedures, there continues to be a need for a surgical mesh that is biocompatible and absorbable, has the ability to withstand the physiological stresses placed on the host collagen, and minimizes tissue erosion, fistulas, or adhesions. Thus, embodiments according to aspects of the present invention provide a biocompatible surgical silk mesh prosthetic device for use in soft and hard tissue repair. Examples of soft tissue repair include breast applications such as breast reconstruction and augmentation, hernia repair, rotator cuff repair, cosmetic surgery, implementation of a bladder sling, or the like. Examples of hard tissue repair, such as bone repair, involve reconstructive plastic surgery, ortho trauma, or the like.

Advantageously, the open structure of these embodiments allows tissue in-growth while the mesh device degrades at a rate which allows for a smooth transfer of mechanical properties to the new tissue from the silk scaffold. According to a particular aspect of the present invention, embodiments employ a knit pattern, referred to as a “node-lock” design. The “node-lock” design substantially prevents unraveling and preserves the stability of the mesh device, especially when the mesh device is cut.

The present invention includes a method of using a knitted silk fabric in a breast reconstruction procedure. This method can comprise the step of implanting (that is placing entirely within the patient's body) a knitted silk fabric in a patient in the vicinity (that is at or adjacent to the anatomical location of the breast reconstruction) of a reconstructed or a to be reconstructed breast of the patient. Typically the silk fabric will be attached to the patient by being sutured in place. The method can further comprising inserting (i.e. implanting) a tissue expander and later the step of removing the tissue expander A breast implant can also be inserted.

In a particular embodiment, a prosthetic device includes a knitted mesh including at least two yarns laid in a knit direction and engaging each other to define a plurality of nodes, the at least two yarns including a first yarn and a second yarn extending between and forming loops about two nodes, the second yarn having a higher tension at the two nodes than the first yarn, the second yarn substantially preventing the first yarn from moving at the two nodes and substantially preventing the knitted mesh from unraveling at the nodes.

In an example of this embodiment, the first yarn and the second yarn are formed from different materials. In another example of this embodiment, the first yarn and the second yarn have different diameters. In further embodiments, wherein the first yarn and the second yarn have different elastic properties. In yet a further example of this embodiment, the at least two yarns are formed from silk.

In another example of this embodiment, a first length of the first yarn extends between the two nodes and a second length of the second yarn extends between the two nodes, the first length being greater than the second length. For instance, the first yarn forms an intermediate loop between the two nodes and the second yarn does not form a corresponding intermediate loop between the two nodes. The first length of the first yarn is greater than the second length of the second yarn.

In yet another example of this embodiment, the first yarn is included in a first set of yarns and the second yarn is included in a second set of yarns, the first set of yarns being applied in a first wale direction, each of the first set of yarns forming a first series of loops at each of a plurality of courses for the knitted mesh, the second set of yarns being applied in a second wale direction, the second wale direction being opposite from the first wale direction, each of the second set of yarns forming a second series of loops at every other of the plurality of courses for the knitted mesh, the first set of yarns interlacing with the second set of yarns at the every other course to define the nodes for the knitted mesh, the second set of yarns having a greater tension than the first set of yarns, the difference in tension substantially preventing the knitted mesh from unraveling at the nodes.

In a further example of this embodiment, the first yarn is included in a first set of yarns and the second yarn is included in a second set of yarns, the first set of yarns and the second set of yarns being alternately applied in a wale direction to form staggered loops, the first set of yarns interlacing with the second set of yarns to define the nodes for the knitted mesh, the alternating application of the first set of yarns and the second set of yarns causing the first set of yarns to have different tensions relative to the second set of yarns at the nodes, the difference in tension substantially preventing the knitted mesh from unraveling at the nodes.

In yet a further example of this embodiment, the first yarn is included in a first set of yarns and the second yarn is included in a second set of yarns, the first set of yarns forming a series of jersey loops along each of a first set of courses for a knitted mesh, the second set of yarns forming a second series of alternating tucked loops and jersey loops along each of a second set of courses for the knitted mesh, the second set of courses alternating with the first set of courses, the second set of yarns having a greater tension than the first set of yarns, the tucked loops of the second set of yarns engaging the jersey loops of the first set of yarns to define nodes for the knitted mesh, the tucked loops substantially preventing the knitted mesh from unraveling at the nodes.

In another particular embodiment, a method for making a knitted mesh for a prosthetic device, includes: applying a first set of yarns in a first wale direction on a single needle bed machine, each of the first set of yarns forming a first series of loops at each of a plurality of courses for a knitted mesh; applying a second set of yarns in a second wale direction on the single needle bed machine, the second wale direction being opposite from the first wale direction, each of the second set of yarns forming a second series of loops at every other of the plurality of courses for the knitted mesh; and applying a third set of yarns in every predetermined number of courses for the knitted mesh, the application of the third set of yarns defining openings in the knitted mesh, wherein the first set of yarns interlaces with the second set of yarns at the every other course to define nodes for the knitted mesh, and the second set of yarns has a greater tension than the first set of yarns, the difference in tension substantially preventing the knitted mesh from unraveling at the nodes.

In yet another embodiment, a method for making a knitted mesh for a prosthetic device, includes: applying a first set of yarns to a first needle bed of a double needle bed machine in a wale direction; applying a second set of yarns to a second needle bed of the double needle bed machine in a wale direction; and applying a third set of yarns in every predetermined number of courses for the knitted mesh, the application of the third set of yarns defining openings in the knitted mesh, wherein the first set of yarns and the second set of yarns are alternately applied to form staggered loops at the first needle bed and the second needle bed, respectively, and the first set of yarns interlaces with the second set of yarns to define nodes for the knitted mesh, the alternating application of the first set of yarns and the second set of yarns causing the first set of yarns to have a different tension relative to the second set of yarns at the nodes, the difference in tension substantially preventing the knitted mesh from unraveling at the nodes.

In a further particular embodiment, a method for making a knitted mesh for a prosthetic device, includes: forming, on a flat needle bed machine, a first series of jersey loops along each of a first set of courses for a knitted mesh; and forming, on the flat needle bed machine, a second series of alternating tucked loops and jersey loops along each of a second set of courses for the knitted mesh, the second set of courses alternating with the first set of courses; wherein the second set of courses has a greater tension than the first set of courses, and the tucked loops along the second set of courses engage the jersey loops of the first set of courses and substantially prevents the knitted mesh from unraveling at the tucked loops. In an example of this embodiment, a continuous yarn forms the first set of courses and the second set of courses. In another example of this embodiment, the first set of courses and the second set of courses are formed by different yarns. In yet another example of this embodiment, the first set of courses and the second set of courses are formed by different yarns having different diameters.

Aspects of the present invention relate to an implantable prosthesis for breast augmentation or reconstruction procedures comprising, a biocompatible and biodegradable fabric structure comprising one or more individual yarns comprised of sericin-extracted native fibroin fibers, wherein the yarn(s) are intertwined to produce the fabric structure, the fabric structure extending in a first dimension and having a first surface adapted to engage and support natural breast tissue or a prosthetic breast implant in a patient. In one embodiment, the fabric structure includes a portion adapted to be fastened to tissue surrounding the chest cavity of the patient. In one embodiment, the fabric structure includes a portion adapted to be fastened to soft tissue surrounding the breast tissue or the prosthetic breast implant. In one embodiment, the fabric structure includes a portion adapted to be fastened to a boney structure adjacent to the breast tissue or the prosthetic breast implant. In one embodiment, the fabric structure is formed in a predefined shape adapted to conform to at least a portion of a region of natural breast tissue or a breast implant. In one embodiment, the predefined shape selected from the group consisting of a circular shape, an oval shape, a crescent shape, a cup shape and an elongated strip. In one embodiment, the fabric structure includes factors for promoting in-growth of breast tissue. In one embodiment, the fabric structure, when implanted, at least partially replaces breast connective tissue. In one embodiment, the fabric structure is formed in an sling shape to provide support for a breast or a breast implant when the fabric structure is implanted in a patient. In one embodiment, the fabric structure is formed in an elongated shape to provide support in an inframammary region of a breast when the fabric structure is implanted in a patient. In one embodiment, the fabric structure is formed in a cup shape to provide inferior support in an inframammary region of a breast when the fabric structure is implanted in a patient. In one embodiment, the fabric structure is formed in a cup shape to provide medial or lateral support for the breast when the fabric structure is implanted in a patient. In one embodiment, the fabric structure is selected from the group consisting of twisted, braided, knitted, stitch bonded, and combinations thereof.

Aspects of the present invention relate to a method of supporting breast tissue or a breast implant in a patient comprising, providing a biocompatible and biodegradable fabric structure comprising one or more individual yarns comprised of sericin-extracted native fibroin fibers, wherein the yarn(s) are intertwined to produce the fabric structure, and inserting the fabric structure between the skin of the patient and the breast tissue or the breast implant. In one embodiment, the method further comprises fastening the fabric structure to tissue surrounding the chest cavity of the patient. In one embodiment, the method further comprises fastening the fabric structure to soft tissue surrounding the breast tissue or the prosthetic breast implant. In one embodiment, the method further comprises fastening the fabric structure to a boney structure adjacent to the breast tissue or the prosthetic breast implant. In one embodiment, the method further comprises forming the fabric structure into a predefined shape adapted to conform to at least a portion of a region of natural breast tissue or a breast implant. In one embodiment, the predefined shape is selected from the group consisting of a circular shape, an oval shape, a crescent shape, a cup shape and an elongated strip. In one embodiment, the method further comprises treating the fabric structure with factors for promoting in-growth of breast tissue. In one embodiment, the fabric structure is inserted in an inframammary region of the breast to provide vertical positioning of the breast and reduce vertical inferior displacement of the breast. In one embodiment, the fabric structure is inserted in a medial side of the breast to provide medial positioning of the breast and reduce medial displacement of the breast. In one embodiment, the fabric structure is inserted in a lateral side of the breast to provide lateral positioning of the breast and reduce lateral displacement of the breast. In one embodiment, the fabric structure is selected from the group consisting of twisted, braided, knitted, stitch bonded, and combinations thereof.

Aspects of the present invention relate to a biocompatible and biodegradable fabric comprising one or more individual yarns comprised of sericin-extracted native fibroin fibers, wherein the yarn(s) are intertwined to produce a fabric structure selected from the group consisting of twisted, braided, knitted, stitch bonded, and combinations thereof. In one embodiment, the fabric is homogeneous. In one embodiment, the fabric has one or more biomechanical properties of connective tissue of the female breast. In one embodiment, the one or more biomechanical properties is selected from the group consisting of ultimate tensile strength, linear stiffness, yield point, percent elongation at break, and combinations thereof. In one embodiment, the fabric is a 2-dimensional mesh. In one embodiment, the connective tissue is superficial fascia or muscular fascia of the female breast subcutaneous fascial system. In one embodiment, the fabric is heterogeneous. In one embodiment, the fabric comprises a 2-dimensional mesh with one or more additional constructs therein. In one embodiment, the 2-dimensional mesh has one or more biomechanical properties of connective tissue of the female breast. In one embodiment, the connective tissue is superficial fascia or muscular fascia of the female breast subcutaneous fascial system. In one embodiment, the additional construct(s) is selected from the group consisting of a twisted construct, a parallel construct, and a braided construct. In one embodiment, the additional construct(s) has one or more biomechanical properties of connective tissue of the female breast. In one embodiment, the connective tissue is selected from the group consisting of fascia mammae, retinaculum fibrosa, and transverse fibrous lamella. In one embodiment, the connective tissue is inframammary retinaculum. In one embodiment, the one or more biomechanical properties is selected from the group consisting of ultimate tensile strength, linear stiffness, yield point, percent elongation at break, and combinations thereof. In one embodiment, the fabric has one or more biomechanical properties of soft tissue within the breast. In one embodiment, the fibroin fibers contain less than 20% sericin by weight. In one embodiment, the fibroin fibers contain less than 10% sericin by weight. In one embodiment, the fibroin fibers contain less than 1% sericin by weight. In one embodiment, one or more of the yarns comprise fibroin fibers that are parallel or intertwined. In one embodiment, one or more of the yarns is a braid, textured yarn, twisted yarn, cabled yarn, or combinations thereof. In one embodiment, one or more of the yarns has a single-level hierarchical organization comprising a group of parallel or intertwined fibers to form the yarn(s). In one embodiment, one or more of the yarns has a two-level hierarchical organization comprising a bundle of intertwined groups, wherein a group comprises parallel or intertwined fibers. In one embodiment, one or more of the yarns has a three-level hierarchical organization comprising a strand of intertwined bundles, wherein a bundle comprises intertwined groups, wherein a group comprises parallel or intertwined fibers. In one embodiment, one or more of the yarns has a four-level hierarchical organization comprising a cord of intertwined strands, wherein a strand comprises intertwined bundles, wherein a bundle comprises intertwined groups, wherein a group comprises parallel or intertwined fibers. In one embodiment, one or more of the yarns comprise a composite of the sericin-extracted fibroin fibers and one or more degradable polymers selected from group consisting of collagens, polylactic acid or its copolymers, polyglycolic acid or its copolymers, polyanhydrides, elastin, glycosamino glycans, and polysaccharides. In one embodiment, the fabric is coated, dobby, laminated, or combinations thereof. In one embodiment, the fabric further comprises a drug. In one embodiment, the fabric further comprises a cell-attachment factor. In one embodiment, the cell-attachment factor is RGD. In one embodiment, one or more of the yarns is treated with gas plasma. In one embodiment, the fabric further comprises biological cells seeded therein.

Aspects of the present invention related to a method for generating connective tissue in the breast of an individual comprising implanting a fabric disclosed herein, wherein the fabric has one or more biomechanical properties of connective tissue, into the individual at an anatomical location within the breast of the individual that provides the appropriate physiologic environment for the development of the connective tissue from the implanted fabric, wherein the fabric is comprised of one or more individual yarns comprised of sericin-extracted native fibroin fibers. In one embodiment, the connective tissue is selected from the group consisting of superficial fascia, muscular fascia, fascia mammae, retinaculum fibrosa, and transverse fibrous lamella. In one embodiment, the fabric is implanted into the individual to replace or repair damaged tissue. In one embodiment, the anatomical location is a site of a surgical incision or of tissue reconstruction. In one embodiment, the fabric is homogeneous. In one embodiment, the fabric is heterogeneous. In one embodiment, one or more of the individual yarns has a hierarchical organization selected from the group consisting of single-level hierarchical organization, two-level hierarchical organization, three-level hierarchical organization, and four-level hierarchical organization.

Aspects of the present invention further relate to a method for supporting a breast structure in an individual comprising implanting a fabric disclosed herein within the breast of the individual in a supporting position relative to the breast structure. In one embodiment, the breast structure comprises native breast tissue. In one embodiment, the breast structure comprises a breast prosthesis. In one embodiment, the breast structure comprises a tissue expander. In one embodiment, the fabric comprises a 2-dimensional mesh. In one embodiment, the fabric further comprises one or more additional constructs therein. In one embodiment, the fabric has one or more biomechanical properties of a connective tissue present naturally in the breast at such a supporting position. In one embodiment, the connective tissue is selected from the group consisting of superficial fascia, muscular fascia, fascia mammae, retinaculum fibrosa, and transverse fibrous lamella.

The present invention also includes a method of using a silk scaffold in breast reconstruction, the method comprising the steps of suturing a knitted, silk scaffold to a chest wall creating a pocket for placement of a tissue expander or a breast implant. This method can further comprise the steps of: inserting the tissue expander; removing the tissue expander; inserting the breast implant, and; inserting the breast implant without a tissue expander. Additionally, the method can also have the step of cutting the silk scaffold to a size to repair a void in an inframammary fold region. The scaffold can be pre-rinsed with antibiotic solution prior to suturing.

The present invention also includes a method of using a silk scaffold in breast reconstruction, the method comprising the steps of: obtaining a knitted scaffold including at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted scaffold from unraveling at the nodes, and; suturing the knitted, silk scaffold to a chest wall creating a pocket for placement of a tissue expander or a breast implant. The first yarn and the second yarn can be formed from different materials. The first yarn and the second yarn can have different diameters and the first yarn and the second yarn can have different elastic properties. In an embodiment of this invention a first length of the first yarn extends between the two nodes and a second length of the second yarn extends between the two nodes, the first length being greater than the second length. Additionally, the first yarn can form an intermediate loop between the two nodes and the second yarn does not form a corresponding intermediate loop between the two nodes, the first length of the first yarn being greater than the second length of the second yarn. Furthermore, the first yarn can be included in a first set of yarns and the second yarn is included in a second set of yarns, the first set of yarns being applied in a first wale direction, each of the first set of yarns forming a first series of loops at each of a plurality of courses for the knitted mesh, the second set of yarns being applied in a second wale direction, the second wale direction being opposite from the first wale direction, each of the second set of yarns forming a second series of loops at every other of the plurality of courses for the knitted mesh, the first set of yarns interlacing with the second set of yarns at the every other course to define the nodes for the knitted mesh, the second set of yarns having a greater tension than the first set of yarns, the difference in tension substantially preventing the knitted mesh from unraveling at the nodes. Further, the first yarn can be included in a first set of yarns and the second yarn is included in a second set of yarns, the first set of yarns and the second set of yarns being alternately applied in a wale direction to form staggered loops, the first set of yarns interlacing with the second set of yarns to define the nodes for the knitted mesh, the alternating application of the first set of yarns and the second set of yarns causing the first set of yarns to have different tensions relative to the second set of yarns at the nodes, the difference in tension substantially preventing the knitted mesh from unraveling at the nodes. Finally, the first yarn can be included in a first set of yarns and the second yarn is included in a second set of yarns, the first set of yarns forming a series of jersey loops along each of a first set of courses for a knitted mesh, the second set of yarns forming a second series of alternating tucked loops and jersey loops along each of a second set of courses for the knitted mesh, the second set of courses alternating with the first set of courses, the second set of yarns having a greater tension than the first set of yarns, the tucked loops of the second set of yarns engaging the jersey loops of the first set of yarns to define nodes for the knitted mesh, the tucked loops substantially preventing the knitted mesh from unraveling at the nodes. The two yarns can be formed from silk, can be approximately 20 to 1000 μm in diameter, and can be substantially constant in diameter.

The present invention also includes a method of using a silk scaffold in a breast augmentation procedure, the method comprising the steps of: (a) implanting a mammary prosthesis into a patient, and; (b) implanting a knitted, silk scaffold adjacent to or abutting the mammary prosthesis in order to support the mammary prosthesis and to facilitate tissue ingrowth at the location of the knitted, silk scaffold. Our invention also encompasses a method for using a silk derived bioresorbable scaffold in a two stage breast reconstruction surgical procedure in a patient that has had a mastectomy, the method comprising the steps of (a) implanting a tissue expander in the patient; implanting a silk derived bioresorbable scaffold in the vicinity of the tissue expander; (c) removing the tissue expander, and; (d) implanting a breast implant in the patient. In this method the breast implant is at stage II implanted into the pocket created by the stage I tissue expander.

The silk scaffold begins to be biodegraded or bioresorbed as soon as it is implanted in a patient. After about one year after implantation into a patient the scaffold has been substantially biodegraded (more than about 50% of the mass of the scaffold has been biodegraded) and the scaffold has become vascularized and shows extensive tissue in growth. After about two years after implantation in a patient at least about 75% of the mass of the scaffold has been biodegraded and after about three years after implantation in a patient at least about 90% to about 100% of the mass of the scaffold has been biodegraded.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1A illustrates the technical back of an example mesh produced on a single needle bed warp knitting machine according to aspects of the present invention.

FIG. 1B illustrates the technical front of the example mesh illustrated in FIG. 1A.

FIGS. 2A-B illustrate an example mesh produced on a double needle bed warp knitting machine according to aspects of the present invention.

FIG. 3 illustrates an example mesh produced with single filament silk yarn according to aspects of the present invention.

FIG. 4 illustrates an example mesh produced on a single needle bed warp knitting machine according to aspects of the present invention.

FIG. 5A illustrates an example mesh produced on a double needle bed warp knitting machine, the example mesh having a parallelepiped pore with a section demonstrating a plush design according to aspects of the present invention.

FIG. 5B illustrates an example mesh produced on a double needle bed warp knitting machine, the example mesh having a hexagonal pore according to aspects of the present invention.

FIGS. 6A-B illustrate example narrow mesh fabrics of varying stitch densities incorporating a plush variation according to aspects of the present invention.

FIG. 7 illustrates an example mesh incorporating loop pile according to aspects of the present invention.

FIG. 8 illustrates an example narrow mesh fabric with pore design achieved through variation in the yarn feed rate according to aspects of the present invention.

FIG. 9A illustrates an example collapsed mesh fabric with hexagonal shaped pores according to aspects of the present invention.

FIG. 9B illustrates an example opened mesh fabric with hexagonal shaped pores according to aspects of the present invention.

FIG. 10 illustrates an example of a stable, non-collapsible, hexagonal-shaped porous mesh fabric according to aspects of the present invention.

FIG. 11A illustrates an example of a three-dimensional mesh with the same technical front and technical back according to aspects of the present invention.

FIG. 11B illustrates the 2.55 mm thickness of the example three-dimensional mesh of FIG. 11A.

FIG. 12 illustrates an example of a three-dimensional mesh with a thickness of 3.28 mm according to aspects of the present invention.

FIG. 13A illustrates the technical front of an example non-porous mesh according to aspects of the present invention.

FIG. 13B illustrates the technical back of the example non-porous mesh of FIG. 13A.

FIG. 13C illustrates the 5.87 mm thickness of the example non-porous mesh of FIG. 13A.

FIG. 14A illustrates an example of a three-dimensional mesh with the same technical front and technical back according to aspects of the present invention.

FIG. 14B illustrates the 5.36 mm thickness of the example three-dimensional mesh of FIG. 14A.

FIG. 15A illustrates the technical front of an example three-dimensional mesh fabric according to aspects of the present invention.

FIG. 15B illustrates the technical back of the example three-dimensional mesh fabric of FIG. 15A.

FIG. 16 illustrates an example mesh produced on a double needle bed weft knitting machine demonstrating shaping of the mesh for a breast support application according to aspects of the present invention.

FIG. 17 illustrates another example mesh produced on a double needle bed weft knitting machine demonstrating shaping of the mesh for a breast support application according to aspects of the present invention.

FIG. 18 illustrates yet another example mesh produced on a double needle bed weft knitting machine demonstrating shaping of the mesh for a breast support application according to aspects of the present invention.

FIG. 19 illustrates a further mesh produced on a double needle bed weft knitting machine demonstrating shaping of the mesh for a breast support application according to aspects of the present invention.

FIG. 20 illustrates another example mesh produced on a double needle bed weft knitting machine demonstrating shaping of the mesh for a breast support application according to aspects of the present invention.

FIG. 21A illustrates a full-thickness rat abdominal defect created using a custom designed 1-cm stainless steel punch, the defect appearing oval in shape due to body wall tension applied.

FIG. 21B illustrates a 4 cm×4 cm example implant centered on top of the open defect of FIG. 21A, and held in place with single interrupted polypropylene sutures (arrow) through the implant and muscle.

FIG. 21C illustrates an explanted specimen 94 days post implantation as shown in FIG. 21B.

FIG. 21D illustrates ball burst testing performed with a 1-cm diameter ball pushed through the defect site reinforced with the mesh according to aspects of the present invention.

FIG. 22 illustrates an example pattern layout for a single needle bed mesh according to aspects of the present invention.

FIG. 23 illustrates an example pattern layout for a single needle bed mesh according to aspects of the present invention.

FIG. 24 illustrates an example pattern layout for a single needle bed mesh according to aspects of the present invention.

FIG. 25 illustrates an example pattern layout for the single needle bed mesh according to aspects of the present invention.

FIG. 26 illustrates an example pattern layout of the double needle bed mesh according to aspects of the present invention.

FIG. 27 illustrates an example pattern layout for the double needle bed weft knitting machine according to aspects of the present invention.

FIGS. 28A and 28B illustrate placement of a silk scaffold in accordance with the present invention.

FIG. 29 illustrates a pocket or sling formed by the scaffold at the pectoralis muscle suitable to hold a tissue expander or breast implant in accordance with the present invention.

FIGS. 30A and 30B are photographs of representative breast reconstruction surgical procedures in accordance with the present invention.

FIGS. 31A, 31B, 31C and 31D are photographs of a representative breast augmentation surgical procedure in accordance with the present invention.

FIG. 32 is a table showing results at plus twelve months (that is at one year follow up after the stage I implantation) from the Example 2 two stage breast reconstruction procedure set forth in Example 2. In the Example 2 study 139 subjects (214 breasts) had stage I implantation of a SERI supported sub-pectoral implanted tissue expander, after mastectomy. At stage II the tissue expander was removed (the SERI was not removed) and replaced by a permanent breast implant. 71 subjects (105 breasts) completed the one year follow up.

### DETAILED DESCRIPTION

All the silk fabrics within the scope of the present invention are knit silk fabrics intended for implantation in a human body. The word “knit” is synonymous with the word “knitted”, so that a knit silk fabric is the same as a knitted silk fabric. The silk fabrics within the scope of the present invention can be warp knit or they can weft knit silk fabrics. Preferably, the silk fabric of the present invention is a biocompatible, warp knit, multi-filament silk fabric. Woven (weaved) silk fabric, woven textiles and woven fabrics are not within the scope of the present invention. A woven material or fabric is made by weaving, which is a process that does not use needles, and results in a fabric with different characteristics. In particular, a woven fabric is made by a non-needle process using multiple yarns that interlace each other at right angles to form a structure wherein one set of yarn is parallel to the direction of fabric formation. On the other hand, a knit fabric is made by using needles (such as for example the needles of a single or double bed knit machine) to pull threads (yarn) up through the preceding thread formed into a loop by the needle, to thereby making the knit fabric (explained in more detail supra). In particular, a knitted fabric is made using needles to have a fabric with one or multiple yarn intermeshing (also referred as interloping). Additionally, non-woven fabrics are also not within the scope of the present invention. Non-woven (also refer to as bonded) fabrics are formed by having multiple fibers cohered together chemically or physically, without use of needles.

Embodiments according to aspects of the present invention provide a biocompatible surgical silk mesh device for use in soft or hard tissue repair. Examples of soft tissue repair include hernia repair, rotator cuff repair, cosmetic surgery, implementation of a bladder sling, or the like. Examples of hard tissue repair, such as bone repair, involve reconstructive plastic surgery, ortho trauma, or the like.

Advantageously, the open structure of these embodiments allows tissue in-growth while the mesh bioresorbs at a rate which allows for a smooth transfer of mechanical properties to the new tissue from the silk scaffold. Furthermore, embodiments employ a knit pattern that substantially prevents unraveling, especially when the mesh device is cut. In particular, embodiments may preserve the stability of the mesh device by employing a knit pattern that takes advantage of variations in tension between at least two yarns laid in a knit direction. For example, a first yarn and a second yarn may be laid in a knit direction to form “nodes” for a mesh device. The knit direction for the at least two yarns, for example, may be vertical during warp knitting or horizontal during weft knitting. The nodes of a mesh device, also known as intermesh loops, refer to intersections in the mesh device where the two yarns form a loop around a knitting needle. In some embodiments, the first yarn is applied to include greater slack than the second yarn, so that, when a load is applied to the mesh device, the first yarn is under a lower tension than the second device. A load that places the at least two yarns under tension may result, for example, when the mesh device is sutured or if there is pulling on the mesh device. The slack in the first yarn causes the first yarn to be effectively larger in diameter than the second yarn, so that the first yarn experiences greater frictional contact with the second yarn at a node and cannot move, or is “locked,” relative to the second yarn. Accordingly, this particular knit design may be referred to as a “node-lock” design.

In general, node-lock designs according to aspects of the present invention employ at least two yarns under different tensions, where a higher tension yarn restricts a lower tension yarn at the mesh nodes. To achieve variations in tension between yarns, other node-lock designs may vary the yarn diameter, the yarn materials, the yarn elastic properties, and/or the knit pattern. For example, the knit pattern described previously applies yarns in varying lengths to create slack in some yarns so that they experience less tension. Because the lower tension yarn is restricted by the higher tension yarn, node-lock designs substantially prevent unraveling of the mesh when the mesh is cut. As such, the embodiments allow the mesh device to be cut to any shape or size while maintaining the stability of the mesh device. In addition, node-lock designs provide a stability that makes it easy to pass the mesh device through a cannula for laparoscopic or arthroscopic surgeries without damaging the material.

Although the node-lock design may employ a variety of polymer materials, a mesh device using silk according to aspects of the present invention can bioresorb at a rate sufficient to allow tissue in-growth while slowly transferring the load-bearing responsibility to the native tissue. Particular embodiments may be formed from Bombyx mori silkworm silk fibroin. The raw silk fibers have a natural globular protein coating known as sericin, which may have antigenic properties and must be depleted before implantation. Accordingly, the yarn is taken through a deplation process. The deplation of sericin is further described, for example, by Gregory H. Altman et al., “Silk matrix for tissue engineered anterior cruciate ligaments,” Biomaterials 23 (2002), pp. 4131-4141, the contents of which are incorporated herein by reference. As a result, the silk material used in the device embodiments contains substantially no sensitizing agents, in so far as can be measured or predicted with standardized biomaterials test methods.

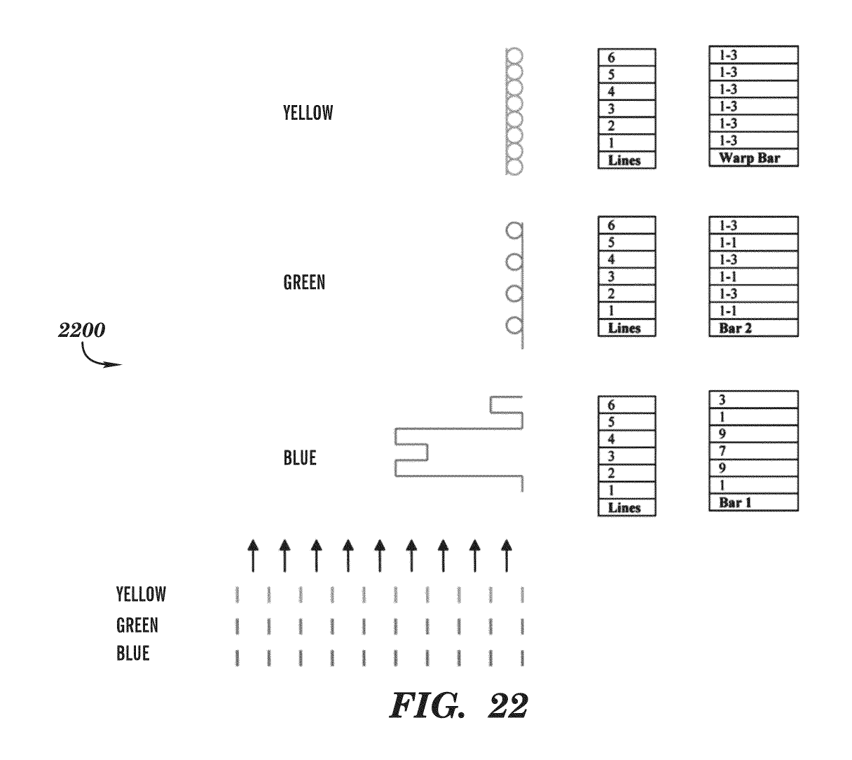

A surgical mesh device according to aspects of the present invention may be created on a single needle bed Comez Acotronic/600-F or a Comez 410 ACO by the use of three movements as shown in the pattern layout 2200 in FIG. 22: two movements in the wale direction, the vertical direction within the fabric, and one in the course direction, the horizontal direction of the fabric. The movements in the wale direction go in opposing directions; a first yarn moving in one direction loops every course while the second yarn moving in the opposite direction loops every other course. The yarns follow a repeated pattern of 3-1 and 1-1/1-3 on a 20 gauge knitting machine, using only half of the needles available on the needle bed. The interlacing of the loops within the fabric allow for one yarn to become under more tension than the other under stress, locking it around the less tensioned yarn; keeping the fabric from unraveling when cut. The other movement within the fabric occurs in every few courses creating the openings in the mesh. These yarns follow a pattern of 1-9/9-7/7-9/9-1/1-3/3-1. These yarns create tension within the fabric when under stress, locking the yarns in the fabric; preventing the fabric from unraveling.

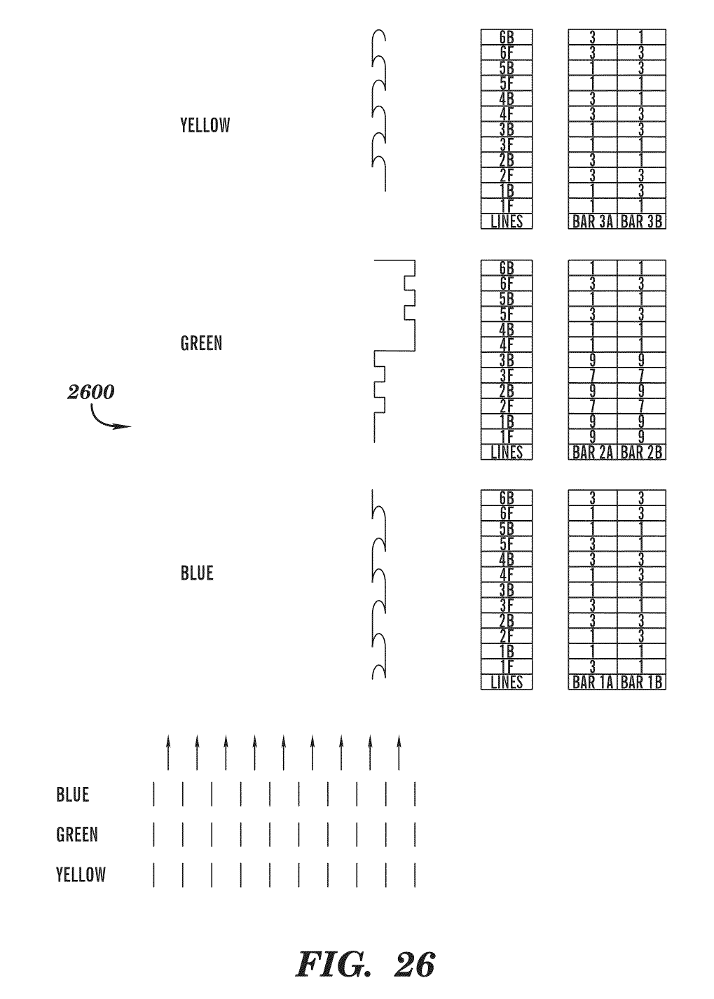

A surgical mesh device according to aspects of the present invention may be created on a double needle bed Comez DNB/EL-800-8B knitting machine by the use of three movements as shown in the pattern layout 2600 in FIG. 26: two movements in the wale direction and one in the course direction. The two movements in the wale direction occur on separate needle beds with alternate yarns; loops that occur in every course movement are staggered within the repeat. The yarns follow a repeated pattern of 3-1/1-1/1-3/3-3 and 1-1/1-3/3-3/3-1. The third movement happens with the yarn that traverses the width of the fabric. The yarn follows the pattern 9-9/9-9/7-7/9-9/7-7/9-9/1-1/1-1/3-3/1-1/3-3/1-1. This fabric is also made at half gauge on a 20 gauge knitting machine and prevents unraveling due to the tension created between the yarns when stressed. The repeat the yarn follows within the pattern is illustrated in FIG. 26.

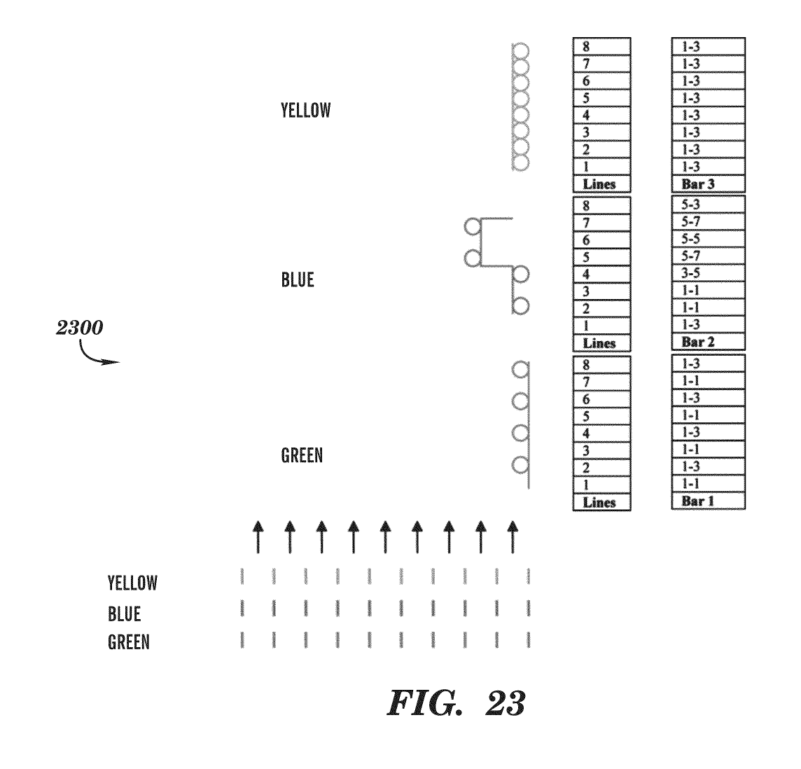

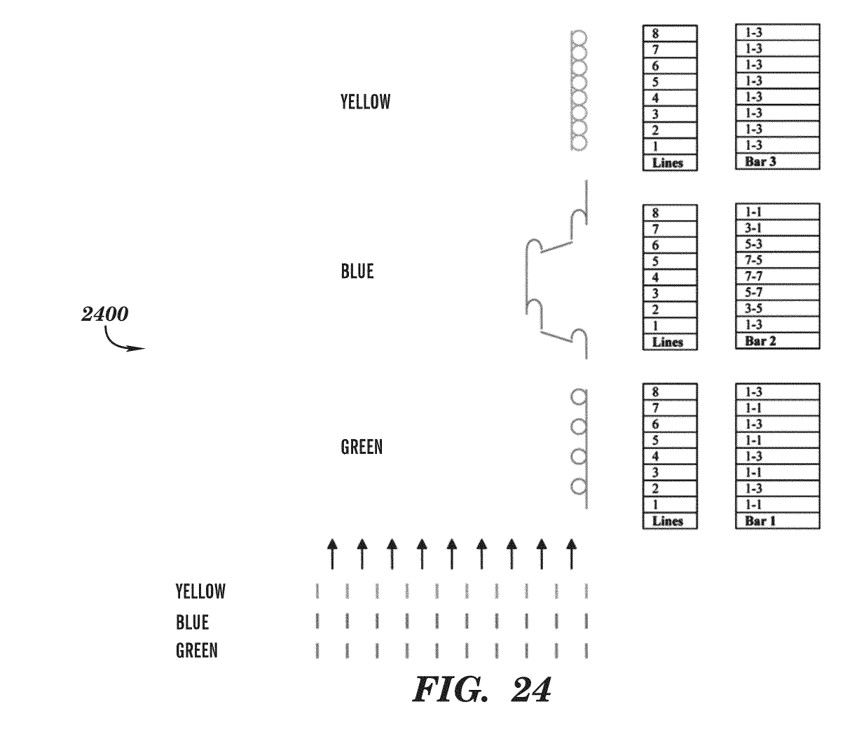

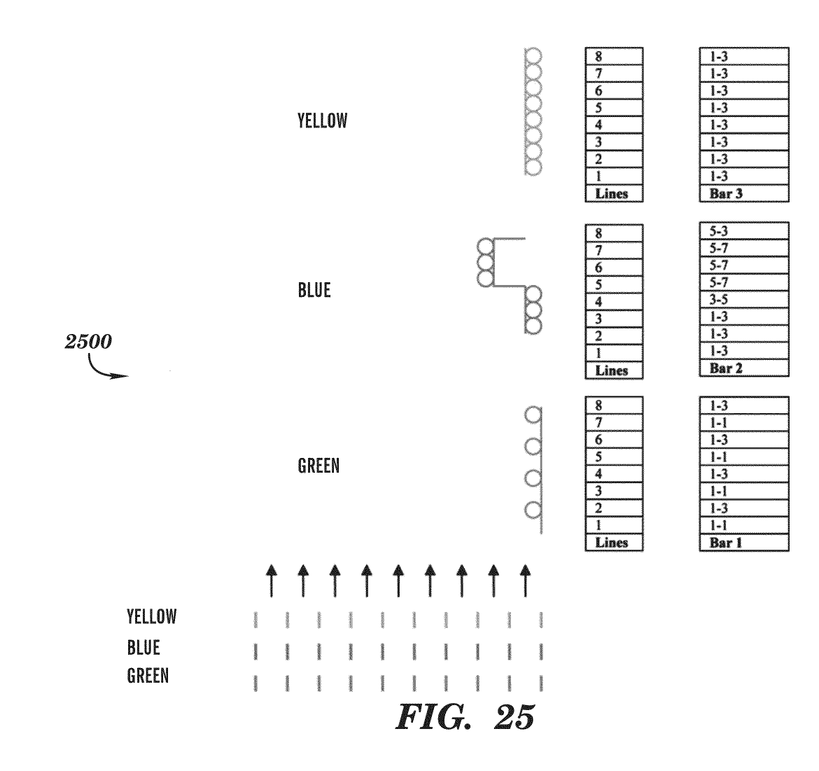

According to the pattern layouts 2300, 2400, and 2500 illustrated in FIGS. 23, 24 and 25, respectively, variations of the surgical mesh pattern are demonstrated for the Single Needle Bed including knitting with an added warp bar in place of using a weft bar insertion. These variations include knitting with the node lock yarns while moving it perpendicularly to one or more wales. These variations may include, but are not limited to, knitting either an open or closed chain stitch in either all or alternating courses. Utilizing a third warp bar, as opposed to a weft bar insertion can also be applied to the double needle warp knitting machine.

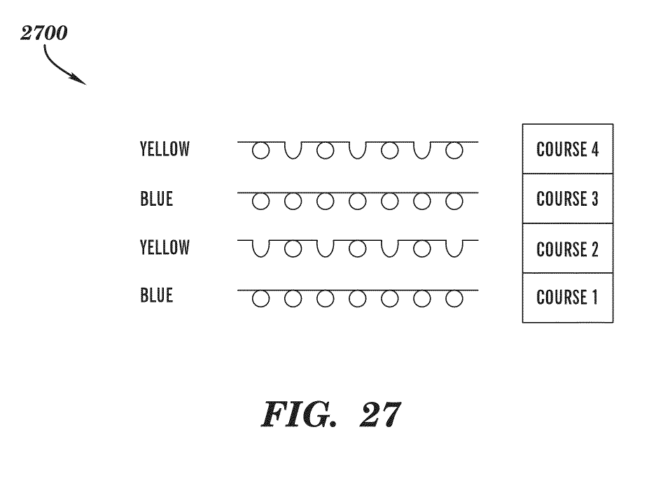

A surgical mesh device according to aspects of the present invention may be formed on the Shima Seiki flat needle bed machine as shown in the pattern layout 2700 in FIG. 27. This knit includes a continuous yarn or at least two different yarn sizes, one of which could be, though not limited to a different material. The knitted mesh would be formed by a regular jersey knit on the first row with loops formed by either a continuous yarn or a yarn of a certain yarn size, while the loops in the second row are formed by tucked loops that occur alternately with jersey knit loops of the same continuous yarn or with a yarn of a different size. The mesh would be shaped during knitting by use of increasing or decreasing stitches; a fashioning technique.

In embodiments employing silk yarn, the silk yarn may be twisted from yarn made by 20-22 denier raw silk fibers approximately 40 to 60 μm in diameter. Preferably, raw silk fibers ranging from 10 to 30 denier may be employed; however any fiber diameters that will allow the device to provide sufficient strength to the intended area are acceptable. Advantageously, a constant yarn size may maximize the uniformity of the surgical mesh mechanical properties, e.g. stiffness, elongation, etc., physical and/or biological properties. However, the yarn size may be varied in sections of the surgical mesh in order to achieve different mechanical, physical and/or biological characteristics in the preferred surgical mesh locations. Factors that may influence the size of the yarn include, but are not limited to: ultimate tensile strength (UTS); yield strength, i.e. the point at which yarn is permanently deformed; percent elongation; fatigue and dynamic laxity (creep); bioresorption rate; and transfer of cell/nutrients into and out of the mesh. The knit pattern layouts 2200, 2300, 2400, 2500, and 2600 illustrated in FIGS. 22-26, respectively, may be knitted to any width limited by the knitting machine width and could be knitted with any of the gauges available with the various crochet machine or warp knitting machine. TABLE 2 outlines the fabric widths that may be achieved using different numbers of needles on different gauge machines. It is understood that the dimensions in TABLE 1 are approximate due to the shrink factor which depends on stitch design, stitch density, and yarn size used.

TABLE 1

| Gauge | Needle Count | Knitting Width |
|---|---|---|
| 48 | 2-5,656 | 0.53-2,997.68 mm |
| 24 | 2-2,826 | 1.06-2,995.56 mm |
| 20 | 2-2,358 | 1.27-2,994.66 mm |
| 18 | 2-2,123 | 1.41-2,993.43 mm |
| 16 | 2-1,882 | 1.59-2,992.38 mm |
| 14 | 2-1,653 | 1.81-2,991.93 mm |
| 12 | 2-1,411 | 2.12-2,991.32 mm |
| 10 | 2-1,177 | 2.54-2,989.58 mm |
| 5 | 2-586 | 5.08-2,976.88 mm |

Embodiments of a prosthetic device according to the present invention may be knitted on a fine gauge crochet knitting machine. A non-limiting list of crochet machines capable of manufacturing the surgical mesh according to aspects of the present invention are provided by: Changde Textile Machinery Co., Ltd.; Comez; China Textile Machinery Co., Ltd.; Huibang Machine; Jakkob Muller AG; Jingwei Textile Machinery Co., Ltd.; Zhejiang Jingyi Textile Machinery Co., Ltd.; Dongguan Kyang the Delicate Machine Co., Ltd.; Karl Mayer; Sanfang Machine; Sino Techfull; Suzhou Huilong Textile Machinary Co., Ltd.; Taiwan Giu Chun Ind. Co., Ltd.; Zhangjiagang Victor Textile; Liba; Lucas; Muller Frick; and Texma.

Embodiments of a prosthetic device according to the present invention may be knitted on a fine gauge warp knitting machine. A non-limiting list of warp knitting machines capable of manufacturing the surgical mesh according to aspects of the present invention are provided by: Comez; Diba; Jingwei Textile Machinery; Liba; Lucas; Karl Mayer; Muller Frick; Runyuan Warp Knitting; Taiwan Giu Chun Ind.; Fujian Xingang Textile Machinery; and Yuejian Group.

Embodiments of a prosthetic device according to the present invention may be knitted on a fine gauge flat bed knitting machine. A non-limiting list of flat bed machines capable of manufacturing the surgical mesh according to aspects of the present invention are provided by: Around Star; Boosan; Cixing Textile Machine; Fengshen; Flying Tiger Machinary; Fujian Hongqi; G & P; Görteks; Jinlong; JP; Jy Leh; Kauo Heng Co., Ltd.; Matsuya; Nan Sing Machinery Limited; Nantong Sansi Instrument; Shima Seiki; Nantong Tianyuan; and Ningbo Yuren Knitting.

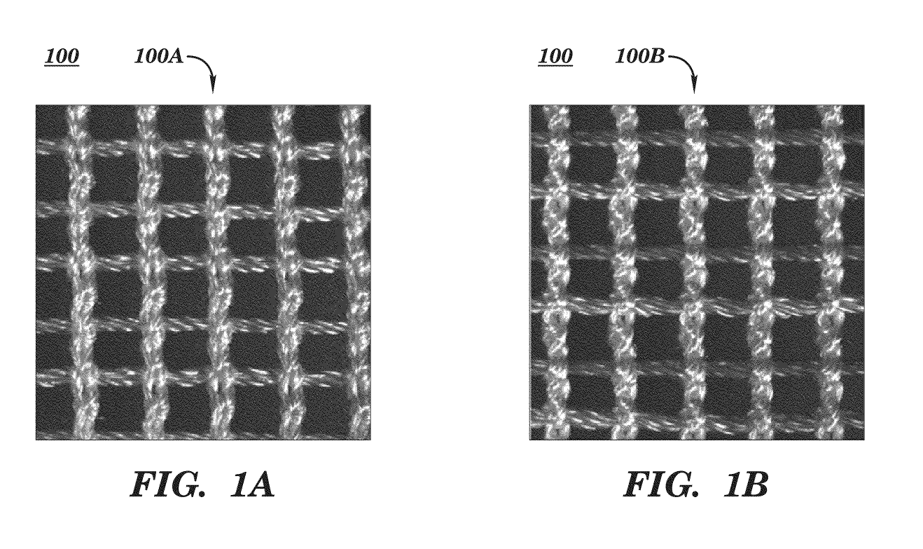

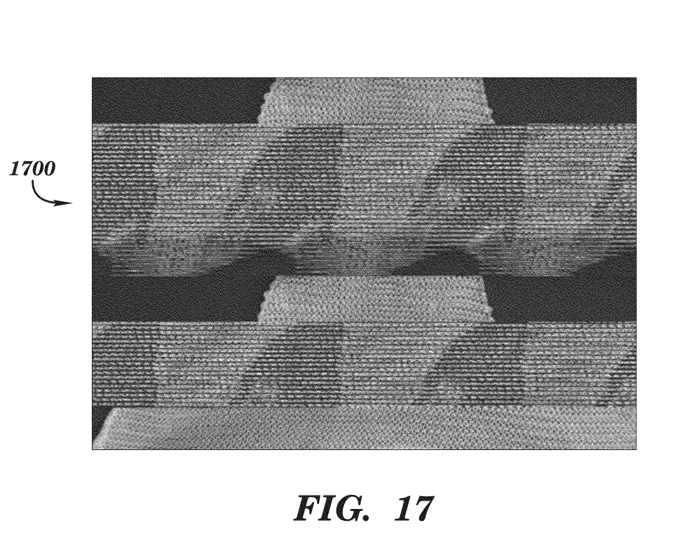

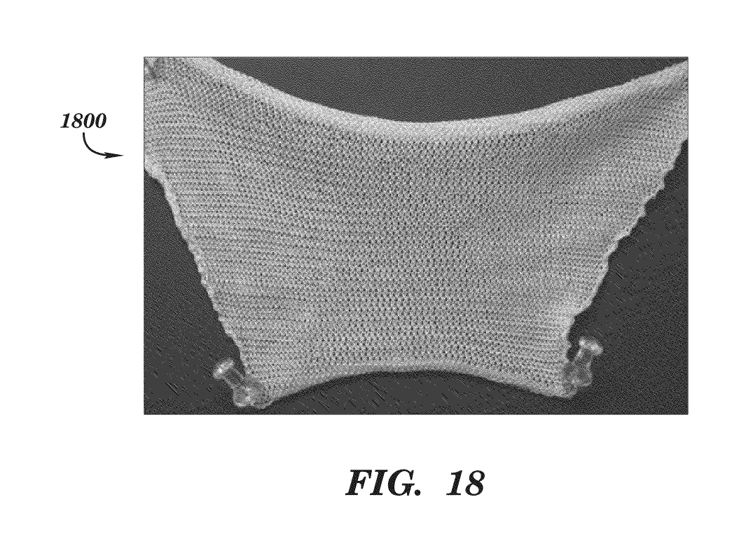

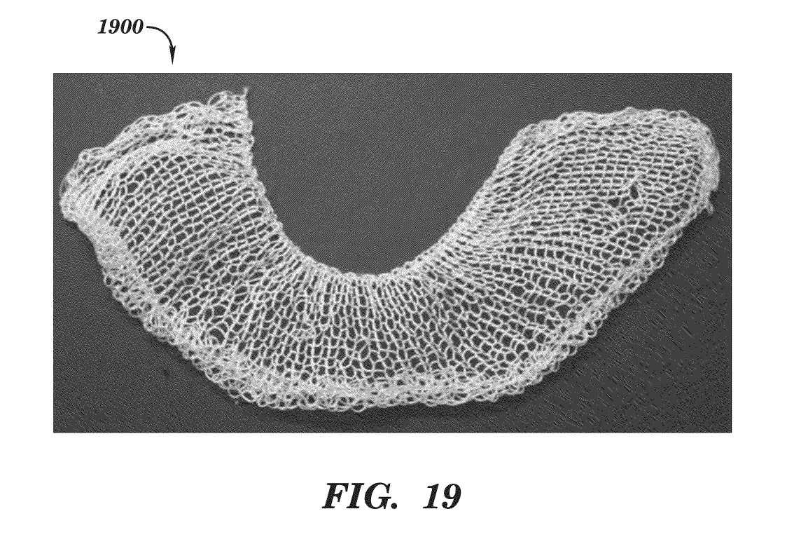

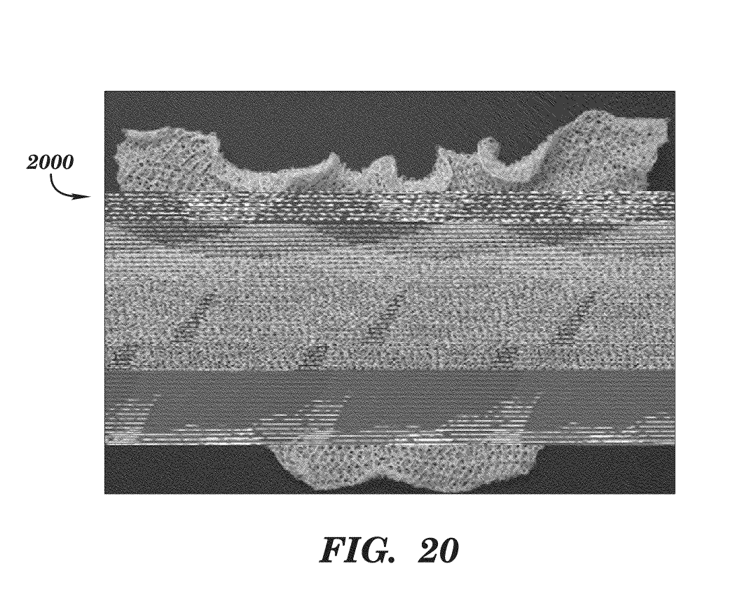

FIGS. 1-20 illustrate example meshes produced according to aspects of the present invention. Referring to FIGS. 1A and B, an example mesh 100 is produced on a single needle bed warp knitting machine according to aspects of the present invention. FIG. 1A shows the technical back 100A of the mesh 100, and FIG. 1B shows the technical front 100B of the mesh 100.

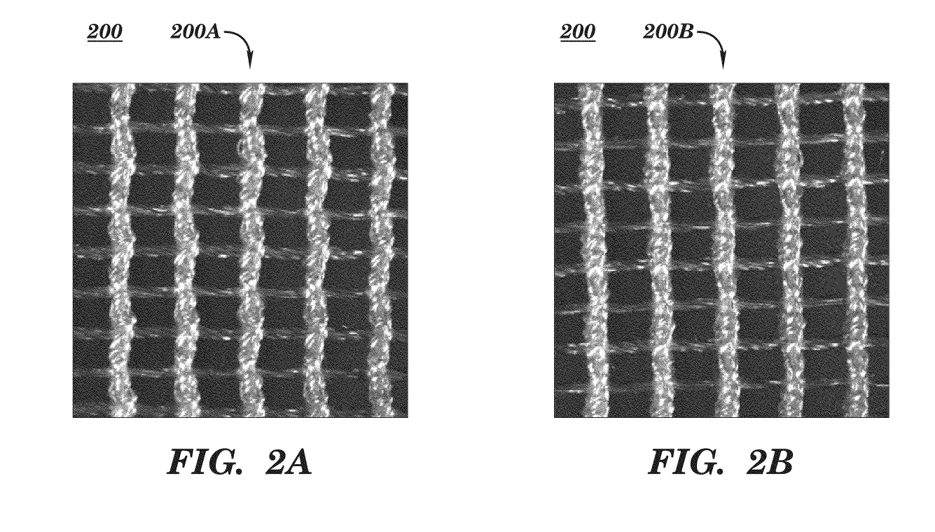

Referring to FIGS. 2A and B, an example mesh 200 is produced on a double needle bed warp knitting machine according to aspects of the present invention. FIG. 2A shows the technical front 200A of the mesh 200, and FIG. 2B shows the technical back 200B of the mesh 200.

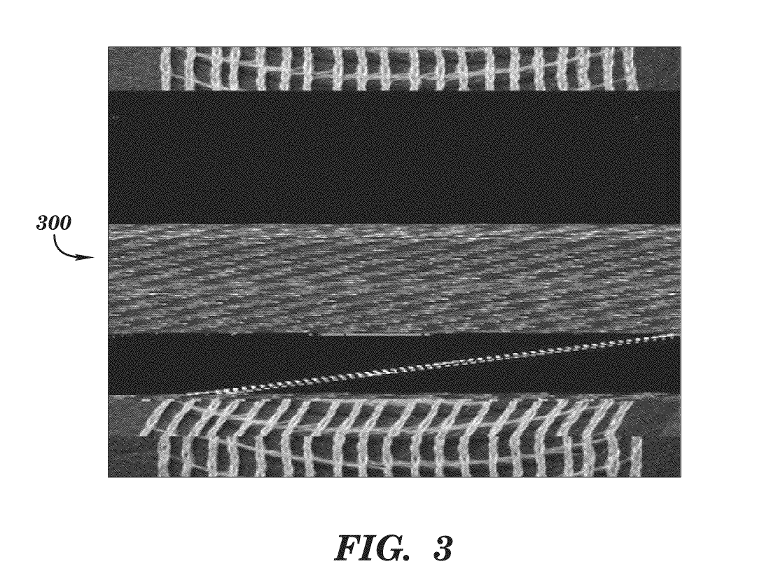

FIG. 3 illustrates an example mesh 300 produced with single filament silk yarn according to aspects of the present invention.

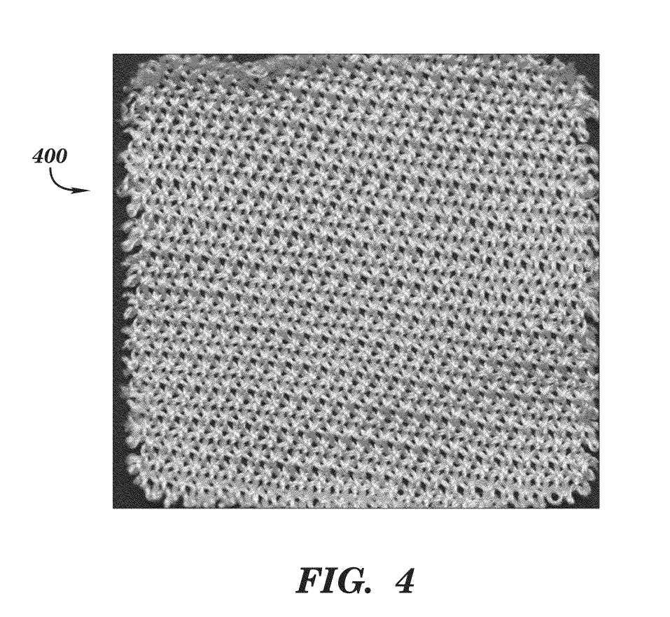

FIG. 4 shows an example mesh 400 produced on a single needle bed warp knitting machine according to aspects of the present invention.

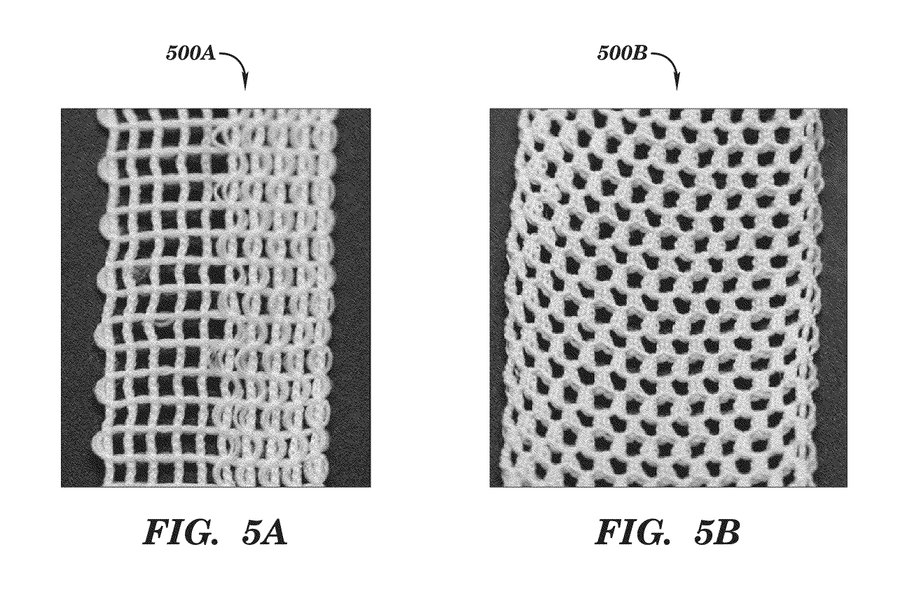

FIG. 5A illustrates an example mesh 500A produced on a double needle bed warp knitting machine. The mesh 500A has a parallelepiped pore with a section demonstrating a plush design according to aspects of the present invention. Meanwhile, FIG. 5B illustrates an example mesh 500B produced on a double needle bed warp knitting machine. The example mesh 500B has a hexagonal pore according to aspects of the present invention.

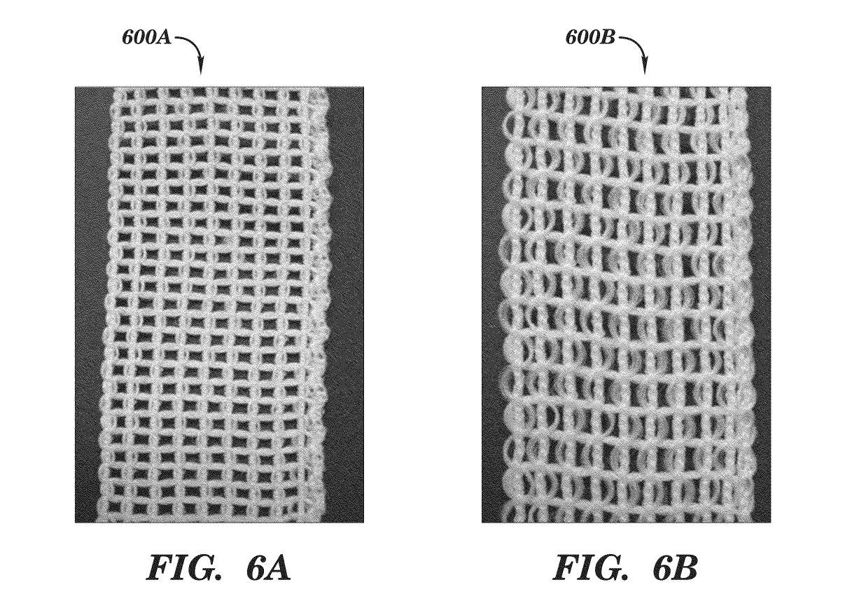

FIGS. 6A and B illustrate example narrow mesh fabrics 600A and 600B according to aspects of the present invention. The mesh fabrics 600A and 600B have varying stitch densities incorporating a plush variation.

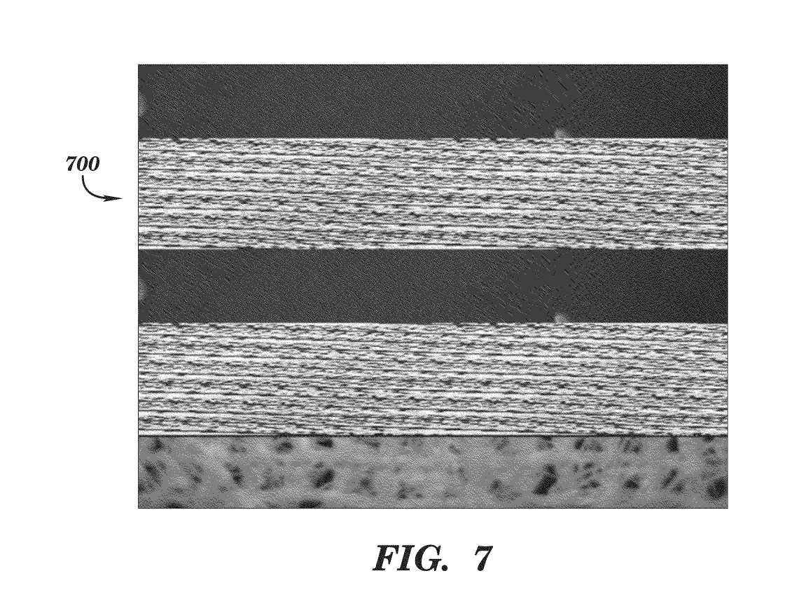

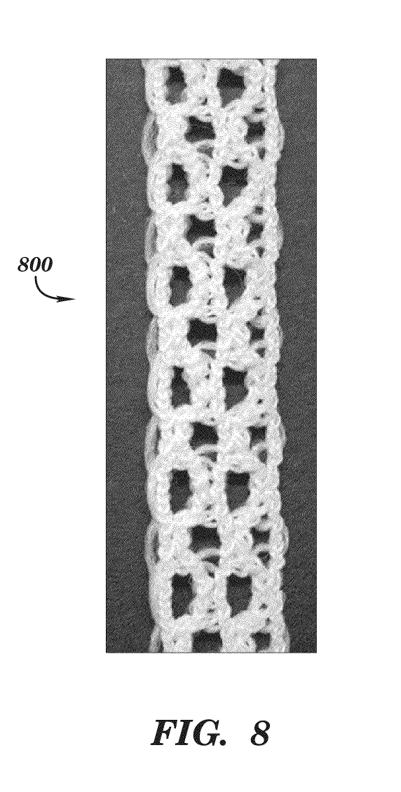

Referring to FIG. 7, an example mesh 700 incorporates loop pile according to aspects of the present invention. FIG. 8 illustrates an example narrow mesh fabric 800 with pore design achieved through variation in the yarn feed rate according to aspects of the present invention.

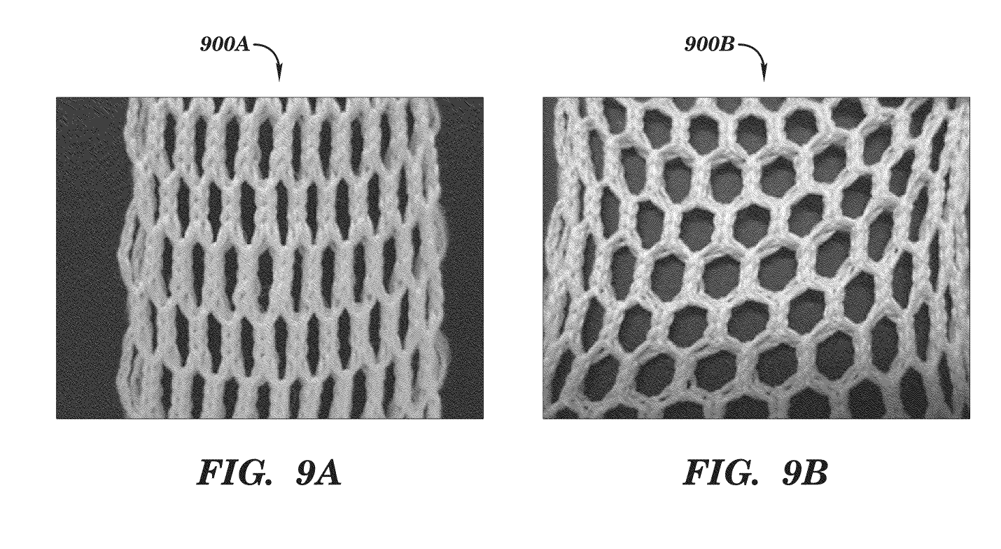

FIG. 9A illustrates an example collapsed mesh fabric 900A with hexagonal-shaped pores according to aspects of the present invention. Meanwhile, FIG. 9B illustrates an example opened mesh fabric 900B with hexagonal shaped pores according to aspects of the present invention.

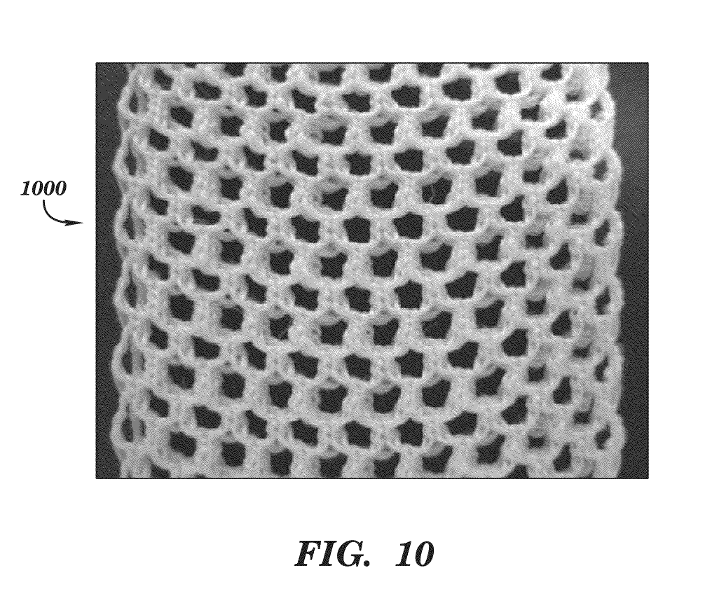

As shown in FIG. 10, an example of a stable, non-collapsible mesh fabric 1000 includes hexagonal-shaped pores according to aspects of the present invention.

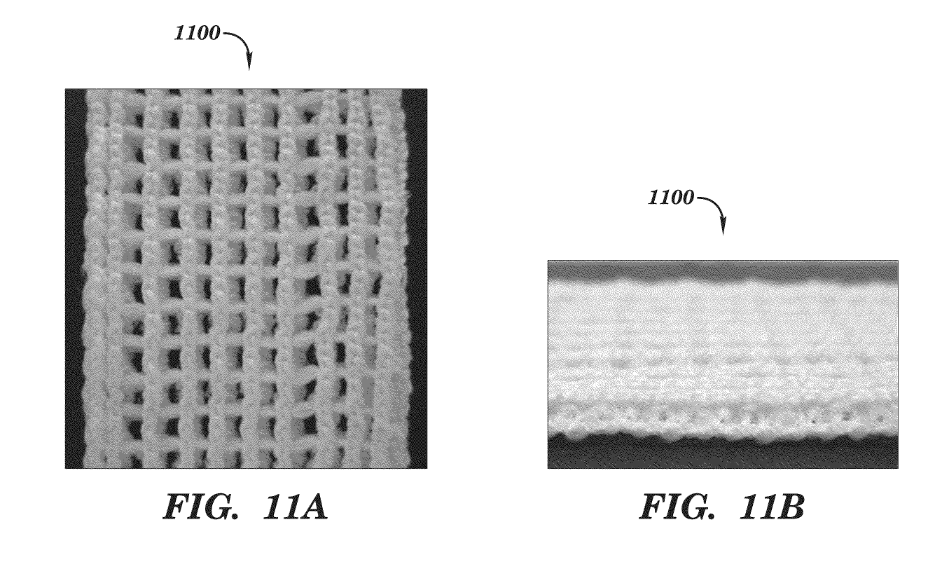

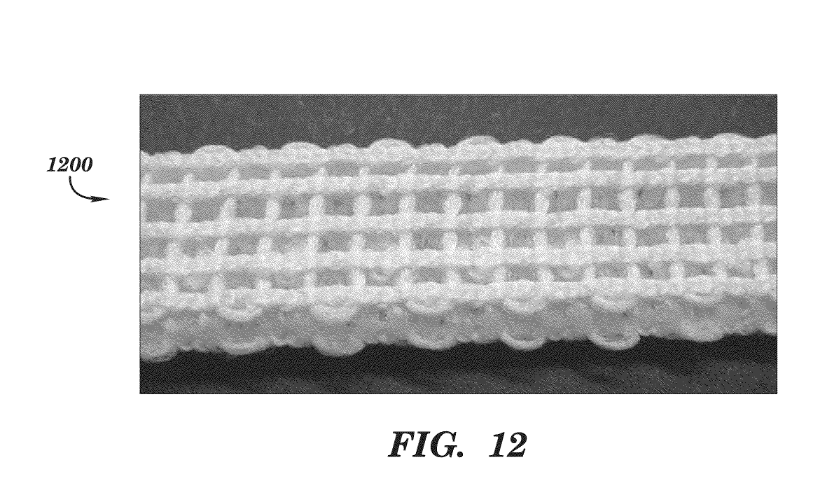

FIG. 11A illustrate an example three-dimensional mesh 1100 with the same technical front and technical back according to aspects of the present invention. FIG. 11B illustrates the 2.55 mm thickness of the three-dimensional mesh 1100. FIG. 12 illustrates another example three-dimensional mesh 1200 with a thickness of 3.28 mm according to aspects of the present invention.

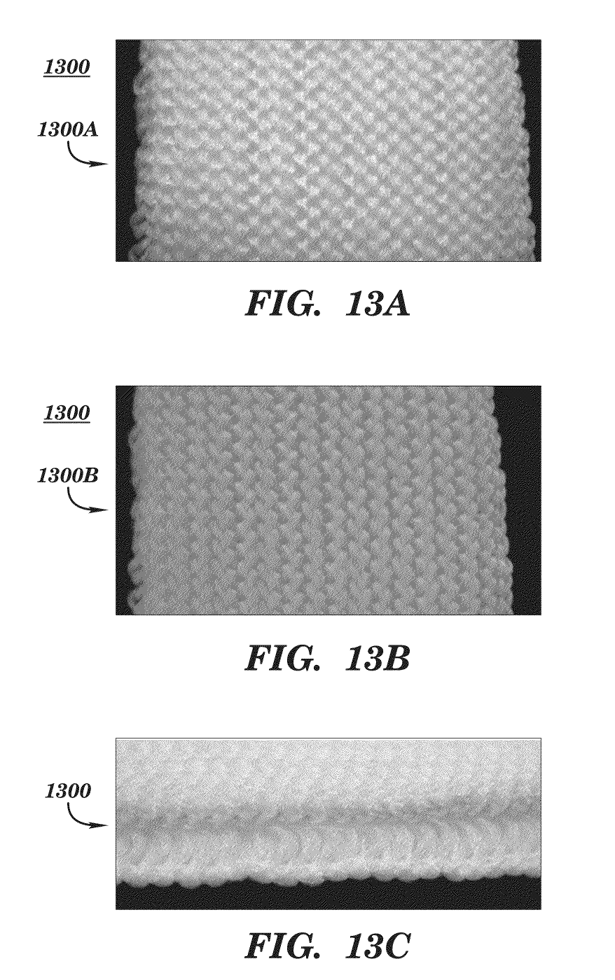

FIGS. 13A-C illustrate an example non-porous mesh 1300 according to aspects of the present invention. FIG. 13A shows the technical front 1300A of the non-porous mesh 1300. FIG. 13B shows the technical back 1300B of the non-porous mesh 1300. FIG. 13C shows that non-porous mesh 1300 has a thickness of 5.87 mm.

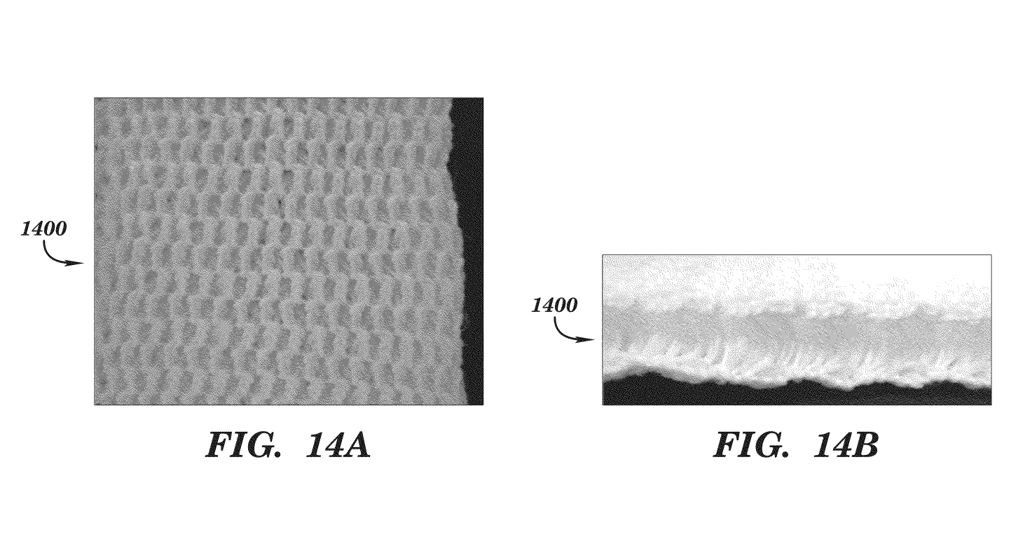

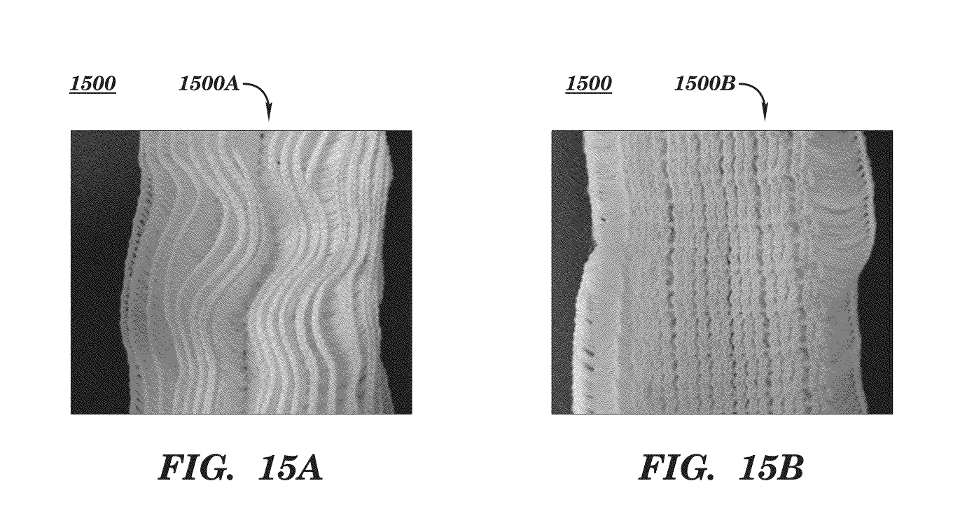

FIG. 14A illustrates an example three-dimensional mesh 1400 with the same technical front and technical back according to aspects of the present invention. FIG. 14B shows that the three-dimensional mesh 1400 has a thickness of approximately 5.36 mm. FIGS. 15A and B illustrate another example three-dimensional mesh fabric 1500 according to aspects of the present invention. FIG. 15A shows the technical front 1500A of the fabric 1500, and FIG. 15B illustrates the technical back 1500B of the fabric 1500.

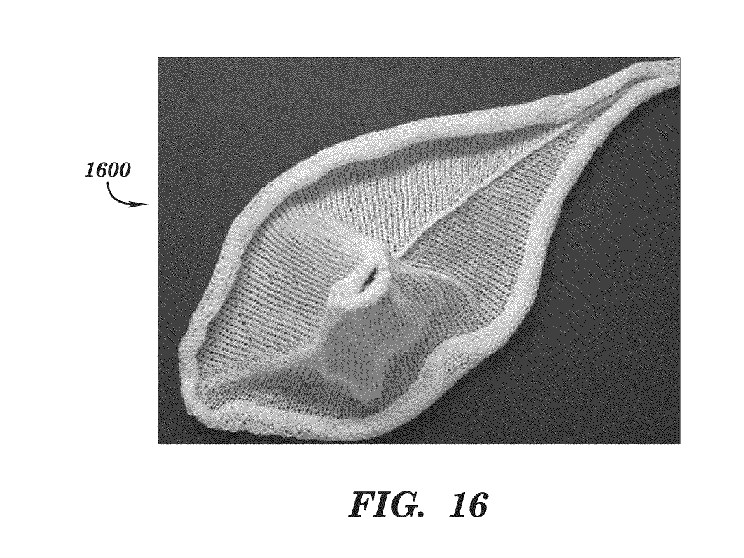

FIGS. 16-20 illustrate respective example meshes 1600, 1700, 1800, 1900, and 2000 that are produced on a double needle bed weft knitting machine. The meshes 1600, 1700, 1800, 1900, and 2000 demonstrate shaping of a mesh for a breast support application according to aspects of the present invention.

A test method was developed to check the cutability of the surgical mesh formed according to aspects of the present invention. In the test method, the surgical mesh evaluated according to the number of were needed to cut the mesh with surgical scissors. The mesh was found to cut excellently because it took one scissor stroke to cut through it. The mesh was also cut diagonally and in circular patterns to determine how easily the mesh unraveled and how much it unraveled once cut. The mesh did not unravel more than one mode after being cut in both directions. To determine further if the mesh would unravel, a suture, was passed through the closest pore from the cut edge, and pulled. This manipulation did not unravel the mesh. Thus, the surgical mesh is easy to cut and does not unravel after manipulation.

Embodiments may be processed with a surface treatment, which increases material hydrophilicity, biocompatibility, physical, and mechanical properties such as handling for ease of cutting and graft pull-through, as well as anti-microbial and anti-fungal coatings. Specific examples of surface treatments include, but are not limited to:

plasma modification

protein such as but not limited to fibronectin, denatured collagen or gelatin, collagen gels and hydrophobin by covalent link or other chemical or physical method

peptides with hydrophilic and a hydrophobic end

peptides contain one silk-binding sequence and one biologically active sequence—biodegradable cellulose

surface sulfonation

ozone gas treatment

physically bound and chemically stabilized peptides

DNA/RNA aptamers

Peptide Nucleic Acids

Avimers

modified and unmodified polysaccharide coatings

carbohydrate coating

anti-microbial coatings

anti-fungal coatings

phosphorylcholine coatings

A method to evaluate the ease of delivery through a cannula was done to make sure the surgical mesh could be used laparoscopically. Various lengths were rolled up and pushed through two different standard sized cannulas using surgical graspers. The mesh was then evaluated to determine if there was any damage done to the mesh. The mesh that was put through the cannulas was found to have slight distortion to the corner that was held by the grasper. The 16 cm and 18 cm lengths of mesh that were rolled up and pushed through the 8 mm cannula had minimal fraying and one distorted pore, respectively. It was also found that no damage was done to the cannula or septum in any of the tests. It was found that appropriately sized surgical mesh will successfully pass through a laparoscopic cannula without damage, enabling its effective use during laparoscopic procedures.

A surgical mesh device according to aspects of the present invention has been found to bio-resorb by 50% in approximately 100 days. In a study by Horan et al., Sprague-Dawley rats were used to compare the bio-resorption of embodiments according to the present invention to Mersilene™ mesh (Ethicon, Somerville, N.J.). The histology reports from the article state that after 94 days, 43% of the initial mesh of the embodiments remained compared to 96% of the Mersilene™ mesh. It was also reported that the in growth was more uniform with the mesh of embodiments than the Mersilene™ mesh. The Mersilene™ was found to have less in growth in the defect region than along the abdominal wall.

Physical properties include thickness, density and pore sizes. The thickness was measured utilizing a J100 Kafer Dial Thickness Gauge. A Mitutoyo Digimatic Caliper was used to find the length and width of the samples; used to calculate the density. The density was found by multiplying the length, width and thickness of the mesh then dividing the resulting value by the mass. The pore size was found by photographing the mesh with an Olympus SZX7 Dissection Microscope under 0.8× magnification. The measurements were taken using ImagePro 5.1 software and the values were averaged over several measurements. The physical characteristics of the sample meshes, including embodiments according to the present invention, are provided in TABLE 2.

TABLE 2

| | Physical Characterization | | |
|---|---|---|---|
| | Thickness | Pore Size | Density |
| Device | (mm) | (mm²) | (g/cm³) |
| Mersilene Mesh | 0.31 ± 0.01 | 0.506 ± 0.035 | 0.143 ± 0.003 |
| Bard Mesh | 0.72 ± 0.00 | 0.465 ± 0.029 | 0.130 ± 0.005 |
| Vicryl Knitted Mesh | 0.22 ± 0.01 | 0.064 ± 0.017 | 0.253 ± 0.014 |
| Present Embodiments - | 1.0 ± 0.04 | 0.640 ± 0.409 | 0.176 ± 0.002 |
| Single Needle Bed (SB) | | | |
| Present Embodiments - | 0.80 ± 0.20 | 1.27 | 0.135-0.165 |
| Double Needle Bed (DB) | | | |

All devices were cut to the dimensions specified in TABLE 3, for each type of mechanical analysis. Samples were incubated in phosphate buffered saline (PBS) for 3±1.25 hours at 37±2° C. prior to mechanical analysis to provide characteristics in a wet environment. Samples were removed from solution and immediately tested.

TABLE 3

| Test Modality | Length (mm) | Width (mm) |
|---|---|---|
| Tensile | 60 | 10 |
| Burst | 32 | 32 |
| Suture Pull-Out | 40 | 20 |
| Tear | 60 | 40 |
| Tensile Fatigue | 60 | 40 |

Ball burst test samples were scaled down due to limitations in material dimensions. The test fixture employed was a scaled (1:2.5) version of that recommended by ASTM Standard D3787. The samples were centered within a fixture and burst with a 10 mm diameter ball traveling at a displacement rate of 60 mm/min. Maximum stress and stiffness were determined from the burst test. Results can be seen in TABLE 4.

TABLE 4

Burst Strength

| Device | Stress (MPa) | Stiffness (N/mm) |
|---|---|---|
| Mersilene Mesh | 0.27 ± 0.01 | 13.36 ± 0.85 |
| Bard Mesh | 0.98 ± 0.04 | 38.28 ± 1.49 |
| Vicryl Knitted Mesh | 0.59 ± 0.05 | 32.27 ± 1.86 |
| Pelvitex Polypropylene Mesh | 0.59 ± 0.04 | 29.78 ± 1.33 |
| Permacol Biologic Implant | 1.27 ± 0.27 | 128.38 ± 22.14 |
| Present Embodiments (SB) | 0.76 ± 0.04 | 46.10 ± 2.16 |
| Present Embodiments (DB) | 0.66 | 40.9 |

Tensile tests were performed along the fabric formation and width axes of each device. A 1 cm length of mesh on each end of the device was sandwiched between pieces of 3.0 mm thick silicone sheet and mounted in pneumatic fabric clamps with a clamping pressure of 70-85 psi. Samples were loaded through displacement controlled testing at a strain rate of 100%/s (2400 mm/min) and or 67%/s (1600 mm/min) until failure. The ultimate tensile strength (UTS), linear stiffness and percent elongation at break can be seen in the following tables. Results can be found in TABLES 5-8. An entry of “NT” indicates that the data has not yet been tested.

TABLE 5

| | Tensile SPTF (Fabric Formation Axis-1600 mm/min) | | | |
|---|---|---|---|---|
| | Strength | Stress | Stiffness | % Elong. @ |
| Device | (N) | (MPa) | (N/mm) | Break |
| Mersilene Mesh | 46.14 ± 3.15 | 10.04 ± 0.71 | 0.90 ± 0.06 | 132.1% ± 9.3% |
| Bard Mesh | 30.90 ± 2.0 | 16.64 ± 1.16 | 3.32 ± 0.26 | 106.5% ± 3.2% |
| Vicryl Knitted | 35.69 ± 3.30 | 35.89 ± 4.48 | 2.59 ± 0.33 | 89.0% ± 7.3% |
| Mesh | | | | |
| Present | 76.72 ± 4.36 | 10.06 ± 0.38 | 7.13 ± 0.50 | 41.5% ± 2.3% |
| Embodiments | | | | |
| (SB) | | | | |
| Present | NT | NT | NT | NT |
| Embodiments | | | | |
| Mesh(DB) | | | | |

TABLE 6

| | Tensile SPTF (Fabric Formation Axis-2400 mm/min) | | | |
|---|---|---|---|---|
| | Strength | Stress | Stiffness | % Elong. @ |
| Device | (N) | (MPa) | (N/mm) | Break |
| Mersilene Mesh | 43.87 ± 5.19 | 14.15 ± 1.68 | 2.18 ± 0.3 | 56.6% ± 3.5% |
| Bard Mesh | 35.29 ± 5.69 | 4.90 ± 0.79 | 0.80 ± 0.23 | 177.3% ± 13.2% |
| Vicryl Knitted | 30.88 ± 3.30 | 14.04 ± 1.50 | 0.76 ± 0.17 | 191.9% ± 14.2% |
| Mesh | | | | |
| Pelvite | 23.05 ± 3.75 | 5.36 ± 0.87 | 0.57 ± 0.07 | 110.0% ± 13.6% |
| Polypropylene | | | | |
| Mesh | | | | |
| Permacol | 164.52 ± 30.58 | 13.71 ± 2.55 | 23.94 ± 2.7 | 23.5% ± 3.3% |
| Biologic Implant | | | | |
| Present | 72.31 ± 7.80 | 6.95 ± 0.75 | 4.31 ± 0.3 | 45.5% ± 5.2% |
| Embodiments | | | | |
| (SB) | | | | |
| Present | 74.62 ± 2.70 | 8.68 ± 0.31 | 4.25 ± 0.13 | 48.3% ± 2.1% |
| Embodiments | | | | |
| (DB) | | | | |

TABLE 7

| | Tensile SPTF (Fabric Width Axis-2400 mm/min) | | | |
|---|---|---|---|---|
| | Strength | Stress | Stiffness | % Elong. @ |
| Device | (N) | (MPa) | (N/mm) | Break |
| Mersilene Mesh | 31.14 ± 2.21 | 10.04 ± 0.71 | 0.90 ± 0.06 | 132.1% ± 9.3% |
| Bard Mesh | 119.80 ± 8.36 | 16.64 ± 1.16 | 3.32 ± 0.26 | 106.5% ± 3.2% |
| Vicryl Knitted | 78.96 ± 9.86 | 35.89 ± 4.48 | 2.59 ± 0.33 | 89.0% ± 7.3% |
| Mesh | | | | |
| Present | 104.58 ± 3.96 | 10.06 ± 0.38 | 7.13 ± 0.50 | 41.5% ± 2.3% |
| Embodiments | | | | |
| (SB) | | | | |
| Present | NT | NT | NT | NT |
| Embodiments | | | | |
| (DB) | | | | |

TABLE 8

| | Tensile SPTF (Fabric Width Axis-2400 mm/min) | | | |
|---|---|---|---|---|
| | Strength | Stress | Stiffness | % Elong. @ |
| Device | (N) | (MPa) | (N/mm) | Break |
| Mersilene Mesh | 28.11 ± 2.93 | 28.11 ± 2.93 | 1.05 ± 0.13 | 128.2% ± 23.6% |
| Bard Mesh | 103.53 ± 8.92 | 14.38 ± 1.24 | 3.43 ± 0.5 | 94.0% ± 8.4% |
| Vicryl Knitted | 106.65 ± 8.46 | 48.48 ± 3.85 | 5.08 ± 0.1 | 58.6% ± 8.4% |
| Mesh | | | | |
| Pelvite | 30.24 ± 5.77 | 7.03 ± 1.34 | 1.48 ± 0.1 | 89.6% ± 9.6% |
| Polypropylene | | | | |
| Mesh | | | | |
| Permacol | 67.71 ± 13.36 | 5.64 ± 1.11 | 8.56 ± 2.0 | 27.4% ± 4.2% |
| Biologic Implant | | | | |
| Present | 98.84 ± 4.79 | 9.50 ± 0.46 | 8.48 ± 0.3 | 39.0% ± 4.1% |
| Embodiments | | | | |
| (SB) | | | | |
| Present | 70.08 ± 2.55 | 8.15 ± 0.30 | 5.87 ± 0.22 | 33.6% ± 2.0% |
| Embodiments | | | | |
| (DB) | | | | |

Tear Strength was found through a method that entailed cutting a 10 mm “tear” into the edge, perpendicular to the long axis edge and centered along the length of the mesh. The mesh was mounted in pneumatic fabric clamps as previously described in the tensile testing methods. Samples were loaded through displacement controlled testing at a strain rate of 100%/s (2400 mm/min) until failure. The load at failure and the mode of failure are shown in TABLE 9.

TABLE 9

| | Tear Strength | |
|---|---|---|
| Device | Strength (N) | Failure Mode |
| Mersilene Mesh | 110.30 ± 5.63 | Tear Failure: 6/6 |
| Bard Mesh | 181.70 ± 12.33 | Tear Failure: 6/6 |
| Vicryl Knitted Mesh | 109.35 ± 4.85 | Tear Failure: 6/6 |
| Pelvitex Polypropylene Mesh | 108.14 ± 6.95 | Tear Failure: 4/6 |
| Permacol Biologic Implant | 273.79 ± 65.57 | Tear Failure: 6/6 |
| Embodiments (SB) | 194.81 ± 9.12 | Tear Failure: 6/6 |
| Embodiments (DB) | NT | NT |

Tensile fatigue testing was performed on the surgical mesh device according to aspects of the present invention and representative predicate types including Vicryl Mesh and Bard Mesh. Samples were loaded into the pneumatic fabric clamps as previously described in the tensile testing methods above. Samples were submerged in PBS at room temperature during cycling. Sinusoidal load controlled cycling was performed to 60% of mesh ultimate tensile strength. Number of cycles to failure was determined during the cyclic studies and can be seen in TABLE 10, where failure was indicated by fracture or permanent deformation in excess of 200%.

TABLE 10

| | Tensile Fatigue |
|---|---|
| Device | Cycles, 60% UTS |
| Bard Mesh | 6994 ± 2987 |
| Vicryl Knitted Mesh | 91 ± 127 |
| Embodiments (DB) | 1950 ± 1409 |

A method was developed to compare the suture pull out strength of the surgical mesh device according to aspects of the present invention to other surgical mesh on the market. Tested mesh was sutured with three 3.5 mm diameter suture anchors (Arthrex, Naples, Fla.) and secured to 15 pcf solid rigid polyurethane foam. Each device was positioned with the center of the 20 mm width over the center anchor with a 3 mm suture bite distance employed during suturing of the mesh to the 3 anchors. The saw bone was mounted in the lower pneumatic fabric clamp and offset to provide loading along the axis of the device when the device was centered under the load cell. The free end of the mesh was sandwiched between the silicone pieces and placed in the upper fabric clamp with 85±5 psi clamping force. Testing was performed under displacement control with a strain rate of 100%/s (1620 mm/min). Maximum load at break and failure mode can be seen in TABLE 11.

TABLE 11

| | Suture-Pull-Out | |
|---|---|---|
| Device | Strength/Suture [N] | Failure Mode |
| Mersilene Mesh | 13.50 ± 1.65 | Mesh Failure: 6 of 6 |
| Bard Mesh | 28.80 ± 3.39 | Mesh Failure: 6 of 6 |
| Vicryl Knitted Mesh | 12.90 ± 1.30 | Mesh Failure: 6 of 6 |
| Pelvitex Polyproplene Mesh | 18.29 ± 4.04 | Mesh Failure: 6 of 6 |
| Permacol Biologic Implant | 47.36 ± 7.94 | Mesh Failure: 6 of 6 |
| Embodiments (SB) | 41.00 ± 2.98 | Mesh Failure: 6 of 6 |
| Embodiments (DB) | 32.57 ± 2.30 | Mesh Failure: 6 of 6 |

By utilizing the pattern for the double needle bed mesh and modifying the yarn size, yarn feed rate and/or needle bed width, the surgical mesh device according to aspects of the present invention would meet the physical and mechanical properties necessary for a soft or hard tissue repair depending on the application. Such properties include pore size, thickness, ultimate tensile strength, stiffness, burst strength and suture pull out. The pore size could be modified dependent to the feed rate to create a more open fabric and the thickness could range from 0.40 mm up to as wide as 19.0 mm. With modifications to the pore size and thickness the UTS, stiffness, burst strength and suture pull out would all be modified as well, most likely tailoring the modifications of the pore size and/or thickness to meet certain mechanical needs.

This mesh, created on the flat knitting machine would be made in such a way to increase or decrease pore size and/or thickness by changing the yarn size and/or changing the loop length found within the knitting settings. The loop placements in combination with the node lock design allow changes to the shape and/or to the mechanical properties of the mesh. A biocompatible yarn with elasticity, such as highly twisted silk, could be used for shaping.

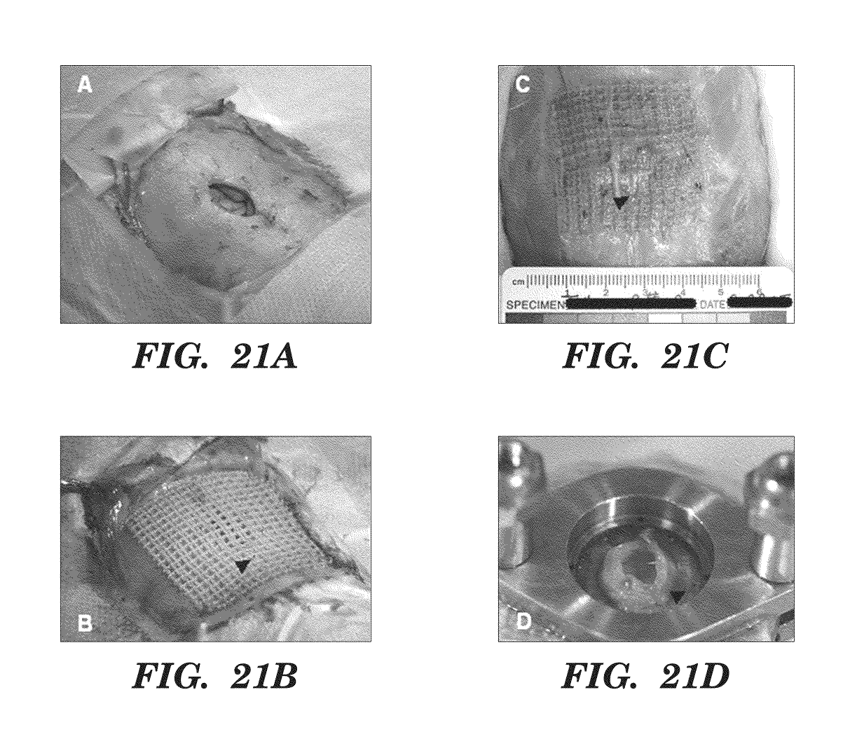

The implantation of a mesh and subsequent testing according to aspects of the present invention is illustrated in FIGS. 21A-D. FIG. 21A illustrates a full-thickness rat abdominal defect created using a custom designed 1-cm stainless steel punch. The defect appears oval in shape due to body wall tension applied. FIG. 21B illustrates a 4 cm×4 cm implant centered on top of the open defect, and held in place with single interrupted polypropylene sutures (arrow) through the implant and muscle. FIG. 21C illustrates an explanted specimen 94 days post implantation. FIG. 21D illustrates ball burst testing performed with a 1-cm diameter ball pushed through the defect site reinforced with the mesh.

While the present invention has been described in connection with a number of exemplary embodiments, and implementations, the present inventions are not so limited, but rather cover various modifications, and equivalent arrangements. For example, a knitted mesh according to aspects of the present invention may be used for a filler material. In one application, the knitted mesh may be cut into 1 mm×1 mm sections to separate one or more nodes, e.g., 3 nodes. The sections may be added to fat tissue or a hydro-gel to form a solution that can be injected into a defective area. Advantageously, the filler material may provide a desired texture, but will not unravel.

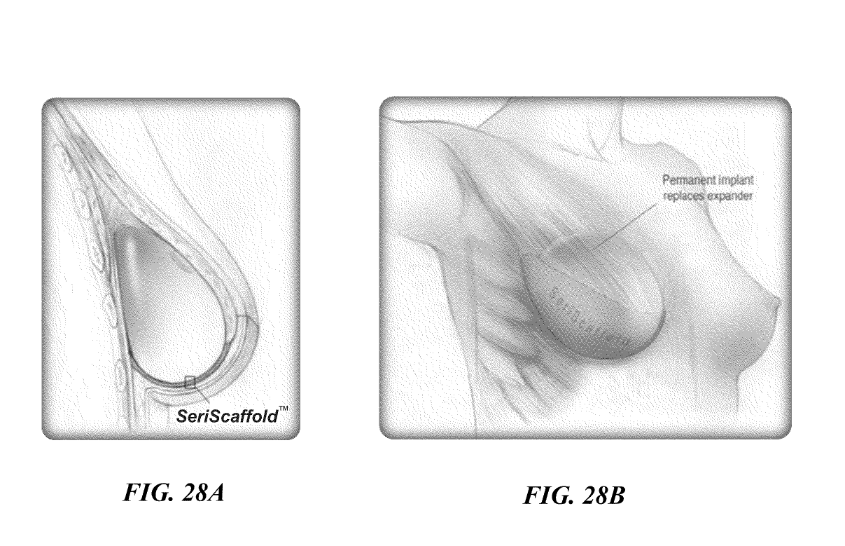

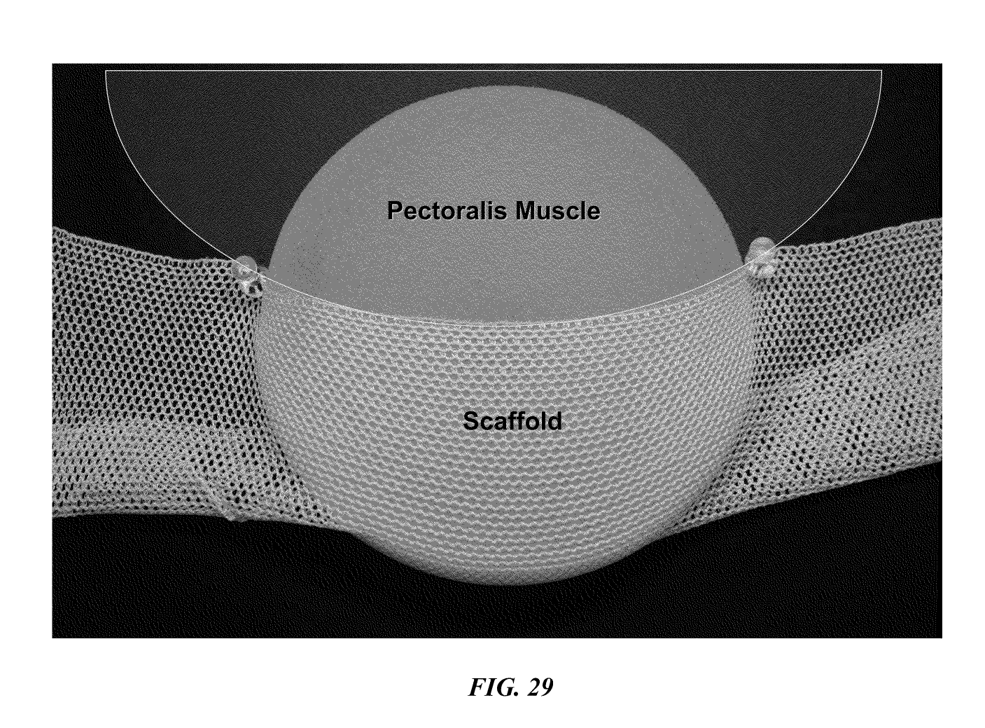

In another aspect of the present invention, the knitted silk mesh or scaffold is used in breast reconstruction procedures. Thus, the present invention relates to method(s) of using the knitted silk scaffold in single-stage or two-stage breast reconstruction. The method comprises suturing the knitted, silk scaffold to a chest wall creating a pocket for placement of a tissue expander or a breast implant. FIGS. 28A and 28B illustrate placement of a silk scaffold in accordance with the methods of the present invention. FIG. 29 illustrates a pocket or sling formed by the scaffold at the pectoralis muscle suitable to hold a tissue expander or breast implant.

In another preferred aspect of the present invention, the silk scaffold is of the node-lock construction such that the knitted mesh or scaffold includes at least two yarns laid in a knit direction and engaging each other to define a plurality of nodes, the at least two yarns including a first yarn and a second yarn extending between two nodes, the second yarn having a higher tension at the two nodes than the first yarn, the second yarn substantially preventing the first yarn from moving at the two nodes and substantially preventing the knitted mesh from unraveling at the nodes. The knitted, silk scaffold has other properties as discussed herein that are particularly desirable in breast reconstruction surgical procedures.

The fabric described herein can be designed for use as an implantable prosthesis in surgical procedures performed to alter the size, shape, position or appearance of a breast mound in a patient. In one embodiment, the fabric described herein is used as an implantable prosthetic device for supporting surrounding tissue and at the same time serving as a scaffold for the in vivo generation of such supportive tissue within the breast of the patient.

As such, the fabric described herein is useful for implantation in procedures such as mastopexy, breast augmentation, and breast reconstruction post-mastectomy.

In one embodiment, the fabric further provides a site for new breast tissue in-growth in vivo.

In one embodiment, the fabric also serves as a scaffold for tissue generation within the breast at the site of implantation. The new tissue generated to replace the fabric can serve as an integral component of the breast repair/augmentation, and/or an aid in recovery from the incisions made during the surgery (e.g., breast reconstruction, breast augmentation, mastopexy). The specific size, shape, and fiber organization of the fabric will vary with respect to the type of procedure and the specific use of the fabric in that procedure, and can be determined by the skilled practitioner for each individual patient. In one embodiment, the fabric is designed so that it can at least partially replace breast connective tissue in the patient (e.g., tissue that was lost due to surgical removal or otherwise damaged).

The fabric can take the form of one or more components designed to resemble and replicate native tissue components within the breast, described herein. The fabric can be designed to replicate one specific tissue structure, or can resemble a plurality of tissue structures (e.g., that are normally found closely associated or interconnected within the breast).

In one embodiment the fabric is designed to replace or replicate connective tissue that spans the breast area and connects the fascia and/or skin (e.g., connective retinaculum, fascia mammae, fibrous lamella). In one embodiment, the fabric is a two-dimensional web or mesh. The web or mesh can be designed to have one or more biomechanical properties of the fascia of the breast (e.g., superficial fascia, muscular fascia).

The fabric in the form of a web or mesh may additionally comprise one or more components which resemble native tissue components within the breast (e.g., ligament or ligament-like structures). For example, a web or mesh with thicker ligament-like structures interspersed through the body of the mesh. Such thicker structures can run along the length of the web, through the center of the web, they can be dispersed in a variety of patterns, e.g., run in straight and/or branching lines radially from the center. They may have circular/elliptical form (e.g., different sized circles arranged to have the same center). In one embodiment, the structures are arranged in a pattern throughout the web that resembles the connective tissue of the breast. Such structures can be designed and generated as integral components of the web or mesh, or can be generated separately and added post production of the web or mesh by attachment. Such structural components of the breast are known in the art.

The fabric is comprised of intertwined yarns (e.g., intertwined by weaving, knitting, or stitch bonding). The yarns are made from sericin-extracted fibroin fibers described herein. The fibroin fibers can be organized into the yarns by one, two, three or four level hierarchical organization, as described herein. For example, parallel or intertwined fibers are grouped together to form the yarn in single-level hierarchical organization. A second level of hierarchical organization is added when a plurality of groups are intertwined together to form one or more bundles present within the yarns. A third level of hierarchical organization is added when a plurality of bundles are intertwined together to form one or more strands present within the yarns. A fourth level of hierarchical organization is added when a plurality of strands are intertwined together to form one or more cords, present within the yarns. Intertwining consists of non-randomly aligning with one another via parallel, helical organization. Such organization can occur at any hierarchical level, to produce a fabric with the desired properties (biomechanical, porosity, etc.). The ordinary skilled artisan will recognize various combinations of these levels of hierarchical organization can be used to produce a fabric with different overall structures (e.g., twisted, braided, knitted, stitch bonded). In addition, fabrics with any combination of these structures can be generated.

In one embodiment, the fabric is designed and implanted to support the breast structure and/or a prosthesis placed within the breast. The structure of the fabric extends in at least one dimension (a first dimension) and has at least one surface (a first surface) adapted to engage the resulting breast (comprising natural breast tissue and/or a prosthetic breast implant). By appropriate placement of the fabric within the patient, the resulting breast structure is shaped by the fabric.

The fabric can be designed to have a variety of different overall shapes (e.g., to conform with the breast tissue when implanted). For example, a fabric that is a web or mesh may be flat, or it may have a concavity. The fabric can have a predefined shape that is adapted to conform to at least a portion of a region of natural breast tissue or of a breast implant, within the patient. In one embodiment, the fabric has a crescent shape, or an elliptical shape. A circular, semi-circular, oval, cup shape, or half-moon shape may also be used. An elongated strip can also be used. In one embodiment, the fabric is sufficiently large to completely or partially cover the lower and/or lateral sections of the breast prosthesis or breast tissue. Such a shape may, for example, allow the fabric to support the lower pole of the breast prosthesis and/or native breast tissue, emulating the inferior and lateral mammary folds. However, the placement of the fabric is not limited to any specific location or alignment within the breast, and will depend upon the specific procedure and ultimate goals of tissue construction. In one embodiment, the fabric is formed in a sling shape (e.g., to provide support for a breast or breast implant, within the patient. In one embodiment, the fabric is formed in an elongated shape (e.g., to provide support in an inframammary region of the breast, within the patient). In one embodiment, the fabric is formed in a cup shape (e.g., to provide medial or lateral support for the breast within the patient).

The fabric may additionally comprise a portion that is adapted to be fastened to the tissue of the patient. This will facilitate implantation. The structure of the portion so adapted will depend upon the means of attachment and/or the place of attachment to the patient. In one embodiment, the attachment is to tissue surrounding the chest cavity of the patient. In one embodiment, the attachment is soft tissue surrounding the breast tissue or surrounding the prosthetic breast implant. In one embodiment, the attachment is to a boney structure adjacent to the breast tissue, or adjacent the prosthetic breast implant.

The fabric may additionally include one or more agents that promote in-growth of cells to thereby generate new breast tissue. Such agents include, without limitation, cell attachment factors, growth factors, attachment promoting materials, drugs, chemoattractants described herein.

### Breast Anatomy

The inframammary fold is the natural boundary of the breast from below where the breast and the chest meet. The inframammary fold is located at the fifth-sixth rib. The lowest portion extends to the sixth intercostal space. This fold has a constant position.

The inframammary region contains a number of thick collagen fibers, stretched between superficial fascia and deep fascia. The superficial fascia is made up of both collagen and elastic fibers. The superficial fascia of the female breast subcutaneous fascial system is exceptionally thick. The superficial fascia connects to the deep fascia (muscular fascia) through thickened retinaculum along the sternum. A connective band known as the anterior breast capsule (fascia mammae) detaches from the superficial fascia. This fascia, and the fascia of the subclavian area support the mammary gland by means of their retinaculum fibrosa (Cooper's ligaments). Cooper's ligaments and also fascia mammae detach from the superficial fascia and connect to the skin (the deep dermis). The inframammary retinaculum originate from the superficial fascia, and consists of merging dense connective retinaculum. The superficial fascia is separated from the muscular fascia through a thin, deep, subcutaneous layer where the connective retinacula are almost horizontal. They are joined by elastic septa which include adipose lobules. In thin women there are only a few of these and they are small, and fixed to the deeper muscular fascia. There is fusion of the superficial fascia with the deeper fascia, along the sternum. Medially the superficial fascia merges into the anterior membrane of the sternum and is composed of fibers coming from the tendinous apparatus of the sternocleidomastoid and pectoralis major muscles.

A transverse fibrous lamella comes off the fascia almost at the 6^{th} rib, and extends the full length of the inframammary crease. This structure has a different texture and a denser consistency from the superficial fascia. Between the superficial and deep fascia, there is a layer consisting of fibroareolar tissue and occasionally fibrofatty tissue. At the submammary area, the tissue is more fibrous at the sixth rib-sixth intercostal space. The superficial fascia connects with the deeper muscular fascia by means of thicker retinaculum at the deep inframammary subcutaneous layer. The superficial fascia here is adherent to the deep plane (muscular fascia) and more resistant to traction. The adherence is histologically made up of multiple, short, fibrous connections which do not pass through the fibromuscular plane.

Mammary ligaments form a circumferential ligament about the breast to form a circumferential fusion between the superficial fascia and the deep fascia. This connective ligament which completely surrounds the breast to form a circular boundary to the cleft between the superficial fascia and deep fascia is often referred to as the circumferential mammary ligament. The circumferential mammary ligament forms a natural boundary connecting two tissue layers that a surgeon dissecting between the layers may use to define and limit the extent of the dissection. These defined layers also offer a region for tissue growth, as disclosed in U.S. Patent Application Publication 2008/0300681.

### Use of the Fabric in Breast Surgery

Fabrics designed to serve as tissue supports and/or scaffolds for breast reconstruction may be used in a wide range of procedures involving breast augmentation or mastopexy, including, for example, in breast lift procedures, breast augmentation procedures, in post-mastectomy reconstruction.

One aspect of the invention relates to the use of the fabric described herein in a method for supporting a breast structure within a patient. The method involves positioning the fabric (e.g., configured as a scaffold for support and new tissue in-growth) within the patient in a supporting position relative to the breast structure. The breast structure may comprise native breast tissue (e.g., a mammary gland), or a breast prosthesis (e.g., a breast implant), or a combination thereof. Generally this involves implanting the fabric structure at an anatomical location between the skin covering the breast tissue and the breast tissue and/or breast implant to be supported within the patient. The specific position (e.g., depth) between the skin and the supported tissue will vary with the actual procedure, and can be determined by the skilled practitioner. In one embodiment, positioning the fabric comprises covering the lower and lateral sections of the breast area. In one embodiment, the fabric is inserted in a medial side of the breast to support medial positioning of the breast and/or implant, and reduce medial displacement of the breast and/or implant. In one embodiment, the fabric is inserted in a lateral side of the breast to support lateral positioning of the breast and/or implant, and reduce lateral displacement of the breast and/or implant. In one embodiment, the fabric comprises one or more biomechanical properties of a tissue that would be present naturally in the breast at such a supportive position. Such tissues are described herein.

Methods for using supportive matrices as surgical tools are well known in the art and can be applied to the fabrics described herein by the skilled practitioner. Implanting the fabric typically involves inserting the fabric structure and fixing the matrix in the desired position. Such methods typically involve fixation of the matrix in the desired position (e.g., across the lower and lateral sections of the breast to support the lower pole of a breast prosthesis/breast tissue, or on the lateral or medial side of the breast to inhibit lateral or medial displacement). Fixation or attachment may be achieved using any suitable method known in the art, for example, by placement of sutures or staples, or with use of a tacking device. Appropriate methods for attachment of the fabric described herein, during the implantation procedure, is to be determined by the skilled practitioner. The fabric described herein can be attached to bone (e.g., one or more ribs), muscle, or soft tissue. In one embodiment, the fabric is attached to one or more soft tissues within the breast region, described herein. Various methods of attachment are known in the art, and include, without limitation, suturing, stapling, gluing, and laying in place. Various attachment methods are described in U.S. Pat. No. 5,584,884.

The exact position of attachment will vary with the specific procedure being performed and can be determined by the skilled artisan. In one embodiment, attachment or fastening of the fabric is to tissue surrounding the chest cavity of the patient. In one embodiment, attachment or fastening is to soft tissue surrounding the breast tissue and/or the prosthetic implant within the patient. The fabric can alternatively be attached or fastened to a boney structure adjacent to the breast tissue and/or the prosthetic implant within the patient.

It may be beneficial or necessary for the skilled practitioner to form the fabric structure into a predefined shape that is adapted to conform to a region, or at least a portion, of the natural breast tissue and/or the prosthetic implant within the patient. Such useful shapes include, without limitation, circular shapes, oval shapes, crescent shapes, cup shapes, and elongated strips.

It may be beneficial to treat the fabric structure with one or more agents that promote cellular in-growth, as described herein.

### Breast Augmentation

In one embodiment, the fabric described herein is used as a surgical tool in breast augmentation. “Breast augmentation” as the term is used herein, refers to increasing the size of a breast, such as is generally achieved by the insertion of prosthetic implants.

The fabric of the instant invention can be used to promote wound healing and soft tissue reconstruction by providing strength and covering at the site of a surgical incision (e.g., at the site of breast implant insertion. It can provide immediate strength to an incision site or site of soft tissue reconstruction/augmentation, and also provide a substrate for new tissue in-growth. In one embodiment, the fabric comprises interconnecting cells or a fibrous network with enough strength to provide closure and protection of incision sites.

In one embodiment, the fabric described herein is used in placement or repositioning of a breast prosthesis. The fabric, for example, can be used to support the lower pole position of breast implants or can be used as a partial or complete covering of the breast implant. Covering of the implants within the fabric provides a beneficial interface with host tissue and reduces the potential for malpositioning or capsular contracture. Covering of the implant also reduces or prevents tissue adhesions to the implant. Ultimately the fabric can be absorbed and replaced by the infiltrating tissue. As such, the fabric can provide temporary scaffolding and well-defined structure until it is no longer needed.

The fabric of the instant invention can be used to reposition a breast implant in follow-up corrective surgery, or can be used prophylactically at the time of initial implant placement to prevent displacement. The fabric can be configured and implanted to position the breast implant in the desired position within the patient (e.g., in completely sub-muscular, partial sub-muscular, or sub-glandular placement).

Implants are typically positioned within the chest in one of three positions: (1) implant over the pectoralis major muscle and under the breast tissue (subglandular); (2) implant partially under the muscle (partial submuscular); and (3) implant completely under the muscle (submuscular). The subglandular placement puts the implant directly behind the breast tissue and mammary gland and in front of the pectoralis major muscle. This placement requires the least complicated surgery and yields the quickest recovery. The downsides of this placement are increased chances for capsular contracture, greater visibility and vulnerability for the implant. This is because only the skin and breast tissue separate the implant from the outside world. Depending on the amount of available breast tissue, the implant may be seen “rippling” through the skin.

Partial submuscular placement involves placing the implant under the pectoralis major muscle. Because of the structure of this muscle, the implant is only partially covered. This alternative reduces the risk of capsular contracture and visible implant rippling, but recovery time from this positioning is typically longer and more painful because the surgeon has to manipulate the muscle during surgery. Also, because of increased swelling, the implant may take longer to drop into a natural position after surgery. Completely submuscular placement puts the implant firmly behind the chest muscle wall. The implant is placed behind the pectoralis major muscle and behind all of the supporting fascia (connective tissue) and non-pectoral muscle groups. This placement has even longer recovery time, potential loss of inferior pole fullness, and involves a more traumatic surgical procedure.

Regardless of location of the implant, in the case of breast augmentation the surgery is carried out through an incision placed to minimize long-term scarring. The incision is made in one of three areas: (1) peri-areolar incision; (2) inframammary fold incision; and (3) transaxillary incision. The peri-areolar incision enables the surgeon to place the implant in the subglandular, partial submuscular or completely submuscular position, with the implant being inserted, or removed, through the incision. Like the peri-areolar incision, the inframammary fold incision provides for all three placement types and both insertion and removal of the implant through the incision. The incision is made in the crease under the breast, allowing for discreet scarring. Once the incision is made, the implant is inserted and worked vertically into place.

Presently, there are very few techniques to reliably maintain the position of implants placed as part of cosmetic or reconstructive surgical procedures. Implant malposition may be the result of several factors, including poor surgical technique, i.e. the implant pocket is too big or too low; implant weight; or lack of soft tissue support. In addition, in reconstructive patients cancer treatments, such as chemotherapy, weaken the soft tissue and surgery, in general, interrupts the natural anatomic plains of the soft tissue. These factors are more profound in patients who have lost excessive amounts of weight. Such situations typically provide extremely poor soft tissue support and the inability of the usual support structures within the breast, such as the inframammary fold, to support the weight of the implant.

In one embodiment, the fabric described herein is implanted within a patient for initial positioning of a breast implant within the patient. In such an embodiment, the fabric may be configured to form a receiving area for receiving the breast implant. The fabric may further comprise one or more regions for tissue affixation. One of the regions may be adapted to attach the fabric to soft tissue surrounding the breast implant or a boney structure within the patient, such as the periosteum of the chest cavity, with a first suture or by conventional or endoscopic tacking.

During implant positioning or repositioning procedures the surgeon can use the initial incision made to insert the fabric, provided the initial incision was peri-areolar or in the inframammary fold, to access and position the implant with the fabric. However, in certain circumstances, such as if the initial incision is in the transaxillary position, it may be necessary to create a new incision. Once the incision is made, the fabric (e.g., rolled up) can be inserted into the body through the incision. The fabric may comprise a suture or tack at the distal end which can be removed enabling the end to unroll once in the desired position for implanting.

The fabric of the present invention can be configured to be implanted within the patient in varying orientations, depending on the specific situation to be remedied or prevented. For example, when used to correct medial displacement (symmastia) or lateral displacement of an implant, the fabric is positioned in a substantially vertical position on the medial or lateral side, respectively, of the implant. When the fabric is used to correct inferior displacement of an implant (otherwise known in the art as bottoming out), the fabric is placed in a substantially horizontal position, supporting the implant from below. Proper positioning of the fabric during the initial implant placement procedure is dependent on the tissue structure surrounding the implant and the desired placement of the implant within the patient.

Fixation of the fabric is achieved, for example, by placement of permanent sutures at key locations via the tissue affixation regions, or with use of a tacking device, either conventional or endoscopic, depending on the placement of the incision. An inframammary fold incision may require suturing of the fabric in place whereas a peri-areolar incision will enable the use of an endoscopic tacking device.

When the fabric is orientated in vertical position to fix or prevent medial displacement of the implant, the fabric can be secured at tissue affixation regions to one or more of the following structures and soft tissue: 1) the backwall to the periosteum of the chest wall, 2) the upper intersection of the first and second portions to the sternal border of the chest wall, 3) at the lower intersection of the first and second portions to periosteum of the chest wall, and 4) on the frontwall to the posterior aspect of the pectoralis fascia.

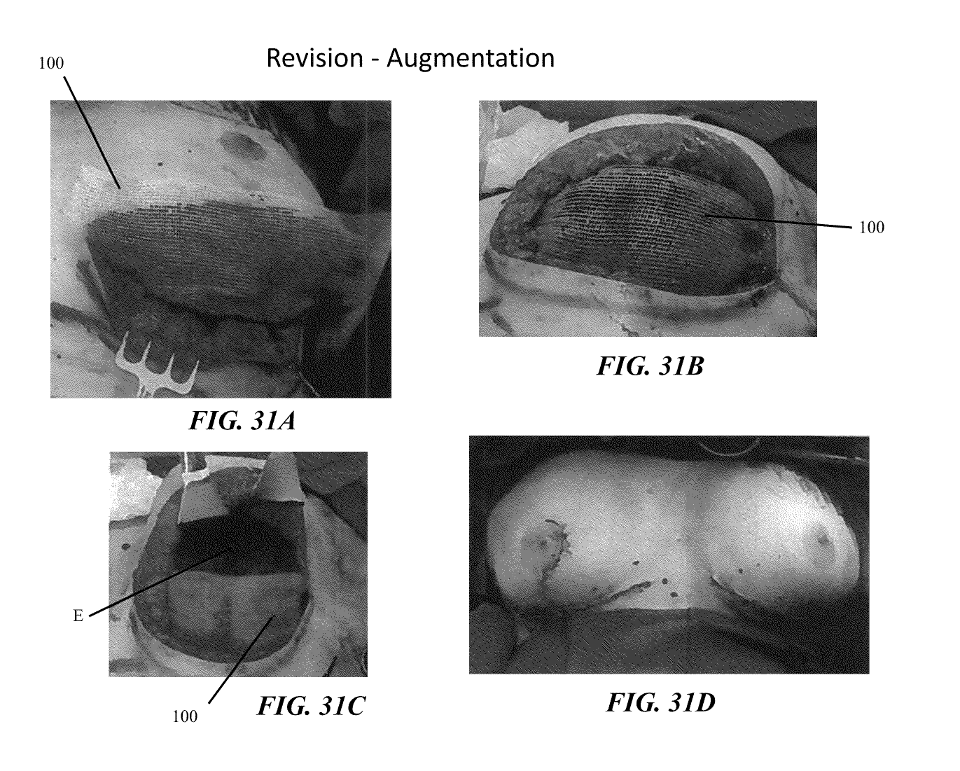

FIGS. 31A, 31B, 31C and 31D are photographs of a representative breast augmentation (revision) surgical procedure in accordance with the present invention. FIG. 31A is a photograph showing a patients' breast cut open, the breast implant having been removed, and SeriScaffold 100 being positioned in the breast. FIG. 31B is a photograph of the FIG. 31A breast showing SeriScaffold 100 further positioned within the breast. FIG. 31C is a photograph of the FIG. 31B breast showing SeriScaffold 100 at its final position in place ready to support a new augmentation breast implant that will be placed at location E. FIG. 31D is a photograph of the FIG. 31C breast (both breasts now shown) after the wound has been sutured closed (over the breast implant supported by SeriScaffold 100), and showing a very positive breast augmentation result.

### Mastopexy (Breast Lift)

Mastopexy, or breast lift, is a procedure designed to improve the appearance of sagging or ptotic breasts. Mastopexy presents one of the greatest challenges to the breast surgeon. Numerous techniques provide improvement in the shape of the breast, but aesthetic improvements comes at the cost of scars. In addition, the use of implants in mastopexy presents specific risks and complications. Four main types of breast lifts exist, crescent mastopexy, donut mastopexy, lollipop or vertical mastopexy and anchor mastopexy, based on the shape of the incision and the resulting scar.

Crescent mastopexy is for patients with mild sagging, excess breast skin in the upper half of the breast, and a normal amount of skin in the lower half, a semi-circular incision is made on the upper portion of the areola. A crescent shaped piece of skin is removed, and when the skin edges are sewn back together, the nipple and areola are raised slightly (1 to 2 inches). A crescent mastopexy is best for women with only mild breast ptosis (sagging).

Donut mastopexy, also called a Benelli mastopexy or circumareolar mastopexy since the incision is around the areola, a donut mastopexy removes a ring of skin from outside the areola. Sutures are then placed around the areola and the skin is tightened like a purse string to lift the breast. Puckering of the skin may occur, and usually resolves on its own within a few months. The donut mastopexy is also useful for women with a projecting nipple/areola complex (sometimes called torpedo or missile shaped breasts), and can also be used to reduce the size of the areola at the same time.

Lollipop or vertical mastopexy, as the name implies, is when an incision for a lollipop mastopexy is made around the areola and then down the center of the breast to the inframammary fold. This technique is used for mild to moderate breast ptosis. As with the circumareolar or donut lift, the size of the areola may be reduced at the same time.

Anchor mastopexy, also referred to as a Wise pattern (or sometimes Weiss pattern) mastopexy, full breast lift, or inverted-T incision, is considered the traditional technique for breast lifting. The incisions are made around the areola, down the center of the lower portion of the breast and then across the breast in the inframammary fold. Like the donut and lollipop incisions, the areola can be made smaller at the same time. The resulting scar is in the shape of an anchor. Although the Wise pattern or anchor mastopexy used to be the standard, it is now usually reserved only for those with moderate to severe breast sagging.

Mastopexy can be performed with or without a corresponding change in the breast size (either breast reduction or breast augmentation).

The fabric described herein can be used in any of these types of procedures. In one embodiment, the fabric described herein is used to promote wound healing and/or tissue support in the procedure. The fabric can be also be used to augment or replace pre-existing breast tissue.

In one embodiment, the fabric is used in a method to reduce breast volume. By way of non-limiting example, the method can be performed as follows:

1. Marking four points on the breast around the areola to determine the amount of skin necessary for both the external skin lining of the new breast and the excess skin in the periareolar region for the dermal flap to be used for the internal skin lining.

2. De-epithelializing the flap to retain the central pedicle.

3. Displace the breast subcutaneous down to the level of the pectoral fascia.

4. Dissecting the skin on the bias in the upper hemisphere in order to progressively increase the thickness of the subcutaneous fat tissue close to the skin.

5. Resecting a central wedge of tissue and shortening the upper hemisphere ray.

6. Dissecting the skin from the parenchymal tissue in the lower hemisphere of the breast.

7. Optionally resecting a second central wedge of tissue in the lower hemisphere.

8. Applying the appropriately shaped fabric over the dermal flap in the lower hemisphere to sling the underside of the breast.

9. Suturing the fabric to the pectoralis fascia to promote elevation and shape of the mammary cone.

10. Suturing closed the external skin lining while fixing the areolar skin to the external skin lining.

11. Dressing the breast in a supportive way that allows drainage of exudates.

In another embodiment, the fabric is used in a method to lift breast tissue. By way of non-limiting example, the method can be performed as follows:

1. Marking four points on the breast around the areola to determine the amount of skin necessary for both the external skin lining of the new breast and the excess skin in the periareolar region for the dermal flap to be used for the internal skin lining.

2. De-epithelializing the flap to retain the central pedicle.

3. Displace the breast subcutaneous down to the level of the pectoral fascia.

4. Dissecting the skin on the bias in the upper hemisphere in order to progressively increase the thickness of the subcutaneous fat tissue close to the skin.

5. Dissecting the skin from the parenchymal tissue in the lower hemisphere of the breast.

6. Applying the appropriately shaped fabric over the dermal flap in the lower hemisphere to sling the underside of the breast.

7. Suturing the fabric to the pectoralis fascia to promote elevation and shape of the mammary cone.

8. Suturing closed the external skin lining while fixing the areolar skin to the external skin lining.

9. Dressing the breast in a supportive way that allows drainage of exudates.

In another embodiment, the fabric is used in a method of mastopexy treatment with breast augmentation. By way of non-limiting example, the method can be performed as follows:

1. Inserting a breast implant either under the muscle in a submuscular pocket where the implant is large and the degree of sagging is greater, or under the breast gland in a subglandular pocket if the implant is small.

2. Marking four points on the breast around the areola to determine the amount of skin necessary for both the external skin lining of the new breast and the excess skin in the periareolar region for the dermal flap to be used for the internal skin lining.

3. De-epithelializing the flap to retain the central pedicle.

4. Displace the breast subcutaneous down to the level of the pectoral fascia.

5. Dissecting the skin on the bias in the upper hemisphere in order to progressively increase the thickness of the subcutaneous fat tissue close to the skin.

6. Dissecting the skin from the parenchymal tissue in the lower hemisphere of the breast.

7. Applying a mastopexy prosthesis over the dermal flap in the lower hemisphere to sling other underside of the breast.

8. Suturing the mastopexy prosthesis to the pectoralis fascia to promote elevation and shape of the mammary cone.

9. Suturing closed the external skin lining while fixing the areolar skin to the external skin lining.

10. Dressing the breast in a supportive way that allows drainage of exudates.

### Breast Reconstruction

Breast reconstruction is the reconstruction of one or both breasts of a female patient following the patient's mastectomy. Mastectomy is the most common treatment of localized breast cancer. While breast reconstruction can be performed at the time of mastectomy, the better candidates are those patients who have confirmed elimination of the cancer as sometimes implant materials and reconstruction will interfere with detection of recurrence. Reconstruction usually involves a two stage process. In stage 1 a tissue expander is inserted (implanted) beneath the skin and the pectoralis muscle of the patient. The tissue expander is an air or saline-filled balloon which while implanted in the patient is periodically injected over a number of months with additional saline in order to gradually stretch the skin and muscle, so as to create a space or pocket in which the stage II breast implant can be implanted in the patient after removal of the tissue expander from the patient. When the skin and muscle are sufficiently lengthened, the selected breast implant (either saline or silicone filled) is inserted (implanted in the patient) to provide, as far as possible through this two stage surgical procedure, a natural looking (reconstructed) breast appearance. Typically to retain the implant properly, an additional section of a patient's tissue, an autograft, is used along the lateral side of the breast, usually the latissimus dorsi or abdominus recti. Autograft tissue bears a risk of tissue morbidity and total coverage and support of the breast implant or the tissue expander with the muscle tissue in the mastectomy pocket is a challenge. Without appropriate coverage, the implant can become exposed and reduce cosmetic outcome.

The fabric (synonomously called the “device” or the “scaffold”. An example of the device or the scaffold is the SeriScaffold™ surgical scaffold, available from Allergan, Inc., Irvine, Calif. under the trade name SERI™, also referred to herein as “SBS”) described herein can be used to promote wound healing and/or tissue support in various surgical procedures such as in a two stage breast reconstruction procedure. The fabric can also be also be used to augment or replace pre-existing breast tissue. The fabric can further be used in implant placement as described herein in a breast reconstruction procedure. In one embodiment, the fabric described herein is used in complement or in place of autograft tissue in the breast reconstruction procedure (e.g., to cover and/or support the implant or the expander at the lower breast pole).

In one embodiment, the fabric of the present invention is used to provide strength to breast fascia and/or soft tissue weakened by the mastectomy surgery. During mastectomy, as much of the superficial fascial system in the inframammary fold is preserved as possible. Generally, Cooper's ligaments are cut in the course of the surgery. In one embodiment the fabric of the present invention is used to recreate the inframammary fold following mastectomy. In one embodiment, the fabric of the present invention is designed to have one or more biomechanical properties of the inframammary fold tissue that is damaged during the mastectomy process. This fabric can be implanted at the location of the damaged tissue. Such implanted fabric supports the reconstructed breast and also serves as a scaffold for the generation of new tissue at that site within the body.

In one embodiment, the fabric of the present invention can be used in place of, or in combination with, the omental flap, in postmastectomy breast reconstruction. One such procedure is described by Goes and Macedo (The Surgery of the Breast, Principles and Art, Lippincott Williams & Wilkins, Second Edition, Chapter 52, pages 786-793, 2006).

The following are non-limiting examples of methods of using the knitted, silk scaffold of the present invention, including but not limited to silk scaffold of node-lock design, in breast reconstruction surgical procedures.

### EXAMPLES

In a breast reconstruction procedure, a knitted silk scaffold having a node-lock design is used. The scaffold is draped and made into a sling or pocket for insertion of a breast implant. Observations of the surgeon can include that the scaffold used is easier to use than existing FLEX HD product. It is possible to see through the scaffold which is desirable. It is noted that the scaffold drapes well when in place and can be implanted as a desirable and useful breast support sling.

### Example 1

### Characteristics of a Silk-Derived Medical Device

We have developed a unique, bioresorbable, silk-derived medical device (a silk-derived bioresorbable scaffold or “SBS”), suitable, among other uses, for use as a supporting scaffold in human breast reconstruction surgery. An embodiment of this medical device has the trade name SeriScaffold™. The desired properties (characteristics) of the medical device (i.e. the SBS) include: long-term bioresorbability; utility as a surgical scaffold; easy to use and suture with no unraveling upon trimming; no side specificity; no swelling/shrinking with hydration, and; inter-sample consistency. Additionally, the pore size of fabric or material which constitutes this desired medical device should facilitate transport of fluid and cells to allow native repair of the tissue defect. SeriScaffold™ is an example of an embodiment of the stated SBS that has been developed for providing soft tissue support and which has all the characteristics set forth in this paragraph.

We determined characteristics of the particular SBS SeriScaffold™ including: thickness and density of dry and hydrated samples, pore area (measured using ImagePro® software), cuttability, drapability/conformability, and laparoscopic insertion. Thus we determined that final and ready to use samples of this SBD, in the dry state, had a thickness (mean±standard deviation) of 0.9±0.0 mm and a density of 0.16±0.01 mg/mm³. On hydration (the SBS was hydrated by incubation in phosphate buffered saline for 2 hours at 37° C.) the SBS consistently maintained its dimensions, with only marginal increases in length, width, and thickness. The SBS pore area was 1.3±0.1 mm². Opposing faces of the SBS were identical to each other, and all dimensions were consistent across multiple manufacturing lots. Additionally, the SBS was cut with a single stroke of standard surgical scissors, leaving no observed fraying or unraveling beyond the immediate pore area or yarn. The SBS was determined to be drapable and conformable to an underlying solid shape, and following brief submersion in a saline bath, could be rolled along its length allowing for successful passage through a ⅞ mm laparoscopic cannula.

Thus we determined that the SBS had ease-of-use characteristics as a surgical scaffold for eg soft tissue support including being porous with low density, easily cut, drapable and conformable, and amenable to laparoscopic insertion. The SBS dimensions were precisely tailored, consistent between lots, and did not change considerably with hydration. The area of SBS pores is conducive to supporting tissue ingrowth. These characteristics coupled with its bioresorption profile make SBS suitable for providing soft tissue support.

### Example 2

### Two-Stage Breast Reconstruction

SeriScaffold™ surgical scaffold (a warp knitt, multi-filament, bioengineered, silk mesh or fabric with a “node lock” knit pattern or structure) can be obtained from Allergan Inc., Irvine, Calif. as well as from Allergan Medical (Santa Barbara, Calif. and Medford, Mass.). SeriScaffold™ surgical scaffold is used as a transitory scaffold for soft tissue support and repair in two-stage breast reconstruction to reinforce deficiencies where weakness or voids existed that required the addition of material to obtain the desired surgical outcome. SeriScaffold™ surgical scaffold is supplied sterile in a single-use 10 cm×25 cm size, with one device utilized per breast. The surgical scaffold is placed during each subject's stage I breast reconstruction with a tissue expander placement procedure.

The procedure followed in this Example is in Stage I—Tissue Expander and SeriScaffold™ Surgical Scaffold Placement is as follows. SeriScaffold™ surgical scaffold is prepared and used in accordance with the supplied package insert and standard-of-care for breast reconstruction procedures. Following mastectomy (either immediate or delayed), the surgical site is readied for subpectoral tissue expander insertion in accordance with standard surgical methods. The serial/lot numbers of the Allergan Natrelle® Style 133V tissue expander and SeriScaffold™ surgical scaffold are recorded. The tissue expander is rinsed in antibiotic solution (according to standard of care) and inserted into the subpectoral pocket. The SeriScaffold™ surgical scaffold is cut to size (prior to, during, and/or after suturing) to repair the void between the pectoral muscle and the chest wall (i.e., inframammary fold region). The SeriScaffold™ surgical scaffold is rinsed with antibiotic solution and sutured in place, with a minimum suture bite of 3 mm or one full row of material. If any cutting is performed in situ, rinsing of the implant site is performed. Intra-operative photography is taken of the scaffold placement prior to closure. The tissue expander is filled as appropriate, drains placed according to usual standard of care and number and location of drain(s) noted. Standard rinsing of the surgical site and closure is performed. Prophylactic antibiotic use and duration is documented.

The surgical drain(s) is removed when deemed appropriate. Tissue expansion is performed in accordance with standard-of-care as appropriate for each subject. The number of fills, volume, and timing of tissue expander fills is recorded at all expansion visits.

The procedure followed in Stage II—Tissue Expander to Breast Implant Exchange is as follows. In a second surgical procedure, the tissue expander is removed and replaced with a breast implant. This procedure is performed as appropriate for each subject, and therefore the duration of elapsed time after the SeriScaffold™ surgical scaffold placement varied between subjects. A standard surgical approach is used to remove the tissue expander. Implant placement is subpectoralis muscle and the pocket is prepared in accordance with standard-of-care. The breast implant is rinsed in antibiotic solution and positioned within the pocket. Standard closure is performed.

SeriScaffold™ surgical scaffold integration is assessed through: (1) scaffold-capsule adherence to tissue expander surface and (2) vascularization of the area. Assessments are recorded during the stage II surgery. Scaffold-capsule adherence to the tissue expander surface and tissue expander adherence to the pectoral muscle are each determined during removal of the tissue expander device in accordance with the following scale: 0, no adherence; 1, minimal adherence (<50% surface area); 2, moderate adherence (50-79% surface area); and 3, complete adherence (80-100% surface area). Capsule vascularization surrounding the biopsy site from the SeriScaffold™ surgical scaffold is visually assessed.

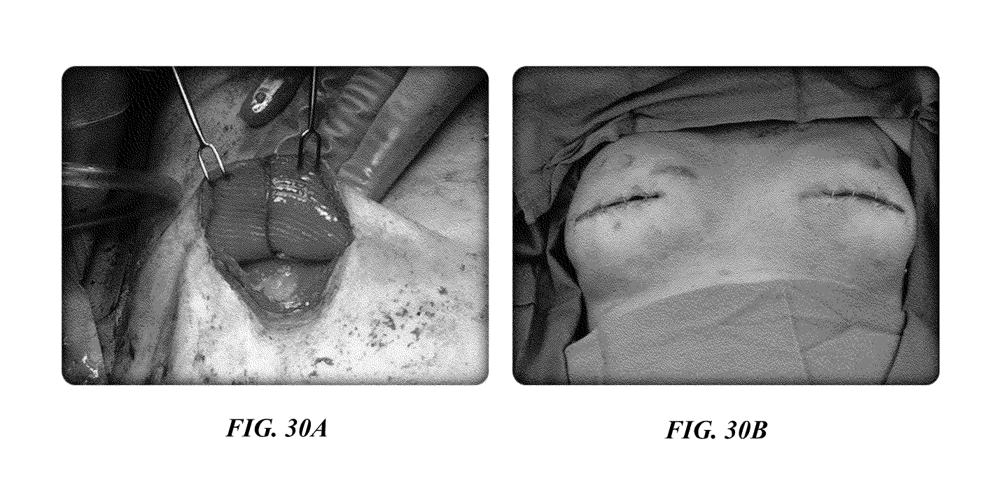

FIGS. 30A and 30B are photographs illustrating a tissue expander to implant exchange performed in the two-stage breast reconstruction surgical procedure in accordance with the present invention. The implant exchange was performed after eleven weeks. The scaffold was integrated and non-palpable. The capsule was flexible and vascularized. The scaffold accommodated expansion to create an ideal breast shape. The result is that the patient has breasts properly positioned and proportioned which look and feel like normal breasts. FIG. 30A is a photograph of a patients breast area tissue cut open, the tissue expander removed, and ready to receive the final breast implant, with the supporting SeriScaffold shown already in place. The SeriScaffold fabric is shown in FIG. 30A in place on the inner surface of the skin flap held open by the two tongs. FIG. 30B is a photograph of the FIG. 30A patient after the breast implant supported by SeriScaffold has been implanted and the wound of each breast so reconstructed sutured closed, and showing a very positive final surgical result.

To summarize results obtained in this Example 2 study: surgeons in breast reconstruction have used an acellular dermis to maintain the position of the pectoralis major muscle and to reinforce the lower pole of the breast mound. These cellular dermis products are cadaver or xenograph tissue-based, and there are published reports of increased risk of complications with their use. We developed and clinically tested the SeriScaffold™ device, a unique silk-derived bioresorbable scaffold (SBS) for soft tissue support in patients undergoing tissue expander-based breast reconstruction.

The SeriScaffold™ device is an FDA-510(k) approved, silk-derived, bioresorbable scaffold (SBS) developed to provide soft tissue support. This Example 2 study (a prospective, single-arm, multicenter clinical trial of patients [“subjects”] without pre-operative or planned post-operative radiation therapy) at the time of mastectomy has been carried out as follows: subjects underwent subpectoral placement of a tissue expander with SBS used to support the lower pole and maintain the position of the pectoralis muscle. In stage 2, the tissue expander was replaced by a breast implant and tissue samples of the integrated scaffold were obtained for histology. These results of the use of a non-cadaver and non-xenograft-derived SBS in 2-stage breast reconstruction showed a high degree of investigator and subject satisfaction, and determined that the SBS was easy to use.

Thus it is noted that, the implanted scaffold (the “SeriScaffold surgical scaffold” or the “device”) provides soft tissue support and facilitates positioning of the implanted tissue expander. The stage I implanted expander is a temporary implant. The stage II breast implant is intended to be a permeant implant, typically remaining implanted in the patient for ten or more years. The scaffold also provides stabilization of the pectoralis muscle and can as well assist with maintenance of the position and appearance of the inframammary fold during stage I of a breast reconstruction and can provide this function after stage I as well. At the time of stage II (when the tissue expander is removed and the breast implant is implanted) the scaffold is then fully or at least partially integrated within the underlying soft tissue which has grown into and around the pores of the scaffold. The scaffold begins to be bioresorbed as soon as the scaffold had been implanted in a patient and the scaffold is completely bioresorbed after about two years after implantation. The scaffold is implanted at stage I to help hold the tissue expander in place (the scaffold is sutured in to form a pocket in which the tissue expander rests and/or the scaffold is draped over the tissue expander). At the time of stage II when the tissue expander is removed and replaced by a breast implant (saline or gel filled) the scaffold is not removed and the scaffold remains in place within the patient.

Preferably, no additional or further scaffold is implanted in the patient at stage II or thereafter. Importantly, the scaffold implanted in the patient in stage I provides soft tissue support and along with the implanted tissue expander maintains the existence of a pocket or space during stage I (the tissue expansion stage) that is until the stage II breast implant implantation in the patient into the pocket or space so maintained during stage I. Thus the scaffold assists to ensure that a pocket or space is available for the placement of the stage II breast implant. Significantly, the stage I implanted scaffold is left in place implanted in the patient and is not removed. By stage II the scaffold has be incorporated into the underlying soft tissue and vasculature has grown in and around it.

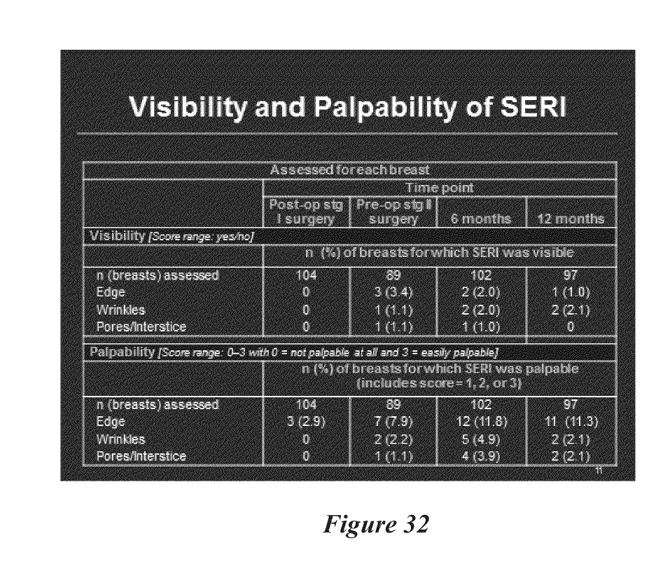

Preferably the scaffold is comprised entirely of or consists essentially of sericin depleted, knitted silkworm silk. FIG. 32 is a table showing results at plus twelve months (that is at one year follow up after the stage I implantation) from this Example 2 two stage breast reconstruction study. In this Example 2 study 139 subjects (214 breasts) had stage I implantation of a SERI supported sub-pectoral implanted tissue expander, after mastectomy. At stage II the tissue expander was removed (the SERI was not removed) and replaced by a permanent breast implant. 71 subjects (105 breasts) completed the one year follow up. As shown by FIG. 32, twelve months after stage I implantation of SERI in a patient, from external evaluation of the patient (that is by observing or manipulating the reconstructed breasts) a visibility or palpability aspect (edge, wrinkle or pores) of the implanted SERI could be determined in from 1% to 11% of the patients in which the SERI had been implanted

The implanted fabric or scaffold (the “SeriScaffold surgical scaffold” or the “device”) begins to be biodegraded or bioresorbed as soon as it is implanted in a patient. The fabric is completely (100%) bioresorbed (biodegraded) within about eighteen months to about 3 years after implantation in a patient.

### Example 3

### Single Stage Breast Reconstruction

SeriScaffold™ silk scaffold is obtained from Allergan Medical for use in breast reconstruction for tissue support and repair in direct-to-implant breast reconstruction surgery. In this Example SeriScaffold™ is used as surgical scaffold in direct-to-implant (DTI), or single-stage, breast reconstruction for soft tissue support and repair. The SeriScaffold™ surgical scaffold is used as a transitory scaffold for soft tissue support and repair to reinforce deficiencies where weakness or voids exist that required the addition of material to obtain the desired surgical outcome.

SeriScaffold™ surgical scaffold is supplied sterile in a single-use 10 cm×25 cm size, with one device utilized per breast. The device is implanted in the subject immediately post mastectomy, during the breast implant placement surgery, in a direct-to-implant breast reconstruction procedure. In this Example SeriScaffold™ surgical scaffold in DTI breast reconstruction is used.

SeriScaffold™ surgical scaffold is prepared and used in accordance with the supplied package insert and standard-of-care for breast reconstruction procedures. Following mastectomy, the surgical site is readied for subpectoral breast implant insertion in accordance with standard surgical methods. The breast implant is rinsed in antibiotic solution and inserted into the subpectoral pocket. The SeriScaffold™ surgical scaffold is optionally cut to size (prior to, during, and/or after suturing) to repair the void between the pectoral muscle and the chest wall (i.e., inframammary fold region). The SeriScaffold™ surgical scaffold is rinsed with antibiotic solution and sutured in place to both the pectoralis muscle and chest wall, with a minimum suture bite of 3 mm or one full row of material. If any cutting was performed in situ, rinsing of the implant site is performed. Drains are placed according to usual standard of care and number and location of drain(s) noted. Rinsing of the surgical site with antibiotic solution and closure is performed. The surgical drain(s) is removed when deemed appropriate. The result is that the patient has breasts properly positioned and proportioned which look and feel like normal breasts.

What is claimed is:

1. A method for using a silk derived bioresorbable scaffold in a two stage breast reconstruction surgical procedure in a patient that has had a mastectomy, the method comprising the steps of:
(a) implanting a tissue expander in the patient;
(c) implanting a silk derived bioresorbable scaffold in the vicinity of the tissue expander;
(c) removing the tissue expander, and;
(d) implanting a breast implant in the patient.

2. The method of claim 1 wherein the breast implant is implanted into a pocket created by the tissue expander.
